(19) United States
(12) Patent Application Publication
(10) Pub. No.: US 2010/0062462 A1
(43) Pub. Date: Mar. 11, 2010

(54) IMMUNODEFICIENCY VIRUSES

(76) Inventors: Andrew D. Badley, Rochester, MN (US); Gary D. Bren, Chatfield, MN (US); Zilin Nie, Rochester, MN (US)

Correspondence Address: FISH & RICHARDSON P.C., PO BOX 1022, MINNEAPOLIS, MN, 55440-1022, US

(21) Appl. No.: 12/097,473

(22) PCT Filed: Dec. 14, 2006
(86) PCT No.: PCT/US06/48101

§ 371 Date: Oct. 23, 2009

(30) Foreign Application Priority Data
Dec. 14, 2005 (US) 60750653

(51) Int. Cl. G01N 33/53; C07K 16/00; C12N 9/96; C12N 5/00; C12Q 1/34

(52) U.S. Cl. 435/7.92; 530/387.9; 530/391.3; 435/188; 435/325; 435/346; 435/18

(57) ABSTRACT

This document relates to methods and materials involved in detecting, monitoring, and prognosing immunodeficiency virus infections. For example, methods and materials for determining whether or not a mammal (e.g., a human) has an immunodeficiency virus infection (e.g., an HIV infection), methods and materials for determining whether or not a mammal with an immunodeficiency virus infection is improving, and methods and materials for determining whether or not a mammal with an immunodeficiency virus infection has an advanced immunodeficiency virus infection are provided.

### STATEMENT AS TO FEDERALLY SPONSORED RESEARCH

This invention was made with government support under grant number AI62261 awarded by National Institutes of Health/National Institute of Allergy and Infectious Diseases. The government has certain rights in the invention.

### BACKGROUND

1. Technical Field

This document relates to methods and materials involved in detecting, monitoring, and prognosing immunodeficiency virus infections in mammals.

2. Background Information

Although multiple stimuli associated with human immunodeficiency virus (HIV) infection are capable of resulting in T cell death, the mechanism by which HIV causes death of infected cells in vivo is unknown. It has been widely believed that bystander mechanisms of cell death are the principal cause of CD4⁺ T-cell depletion in patients with HIV infection. However, recent studies suggest that death of T cells in lymphoid tissues is due to direct cytotoxicity, viral cytopathicity, or lytic infection, rather than bystander cytotoxicity. Indeed, in both HIV-infected patients with acute disease and macaques acutely infected with simian immunodeficiency virus (SW), there is a rapid and profound destruction of virus containing activated memory CD4⁺/CCR5⁺ T cells in the gut that occurs within the first weeks of infection (Brenchley et al., J. Exp. Med., 200:749-59 (2004); Mehandru et al., J. Exp. Med., 200:761-70 (2004); Mattapallil et al., Nature, 434:1093-7 (2005); Li et al., Nature, 434:1148-52 (2005)).

### SUMMARY

This document provides methods and materials related to detecting, monitoring, and prognosing immunodeficiency virus infections. For example, this document provides methods and Materials for determining whether or not a mammal (e.g., a human) has an immunodeficiency virus infection (e.g., an HIV infection), methods and materials for determining whether or not a mammal with an immunodeficiency virus infection is improving, and methods and materials for determining whether or not a mammal with an immunodeficiency virus infection has an advanced immunodeficiency virus infection. As described herein, cells infected with an immunodeficiency virus can express an immunodeficiency virus protease that cleaves a cellular caspase polypeptide (e.g., a caspase 8 polypeptide) to form a fragment (e.g., a casp8p41 polypeptide) of the cellular caspase polypeptide not normally detected in uninfected cells. Thus, the presence of such a fragment (e.g., a casp8p41 polypeptide) in cells from a mammal can indicate that that mammal has an immunodeficiency virus infection. In addition, the level of such a fragment in cells from a mammal can indicate the state (e.g., a mild, moderate, or severe state) of an immunodeficiency virus infection. Having the ability to detect, monitor, and prognose immunodeficiency virus infections can help clinicians provide appropriate medical treatments for infected individuals.

This document also provides methods and materials for assessing the effectiveness of anti-immunodeficiency virus infection treatments. For example, clinicians can determine whether or not an anti-immunodeficiency virus infection treatment is effective by assessing the level of an immunodeficiency virus protease-derived caspase polypeptide (IVP caspase polypeptide). Although not limited to any particular mode of action, an IVP caspase polypeptide can be generated via cleavage of a caspase polypeptide (e.g, a procaspase-8 polypeptide) by an immunodeficiency virus protease (e.g., an HIV protease). A p41 fragment of human caspase 8 (a casp8p41 polypeptide) is an example of an IVP caspase polypeptide. Assessing mammals with an immunodeficiency virus infection using the level of an IVP caspase polypeptide, rather than numbers of CD4⁺ T cells or number of virions, can allow clinicians to determine the effectiveness of therapies sooner.

This document also provides antibodies that can have the ability to bind to an IVP caspase polypeptide at a standard high affinity, while lacking the ability to bind to an uncleaved caspase polypeptide at that same affinity. Such antibodies can be used to detect an immunodeficiency virus infection (e.g., an HIV infection) in a mammal (e.g., a human).

In one embodiment, this document features an antibody preparation comprising an antibody or fragment thereof, where the antibody or the fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide. The antibody can be a monoclonal antibody. The antibody or the fragment thereof can comprise a label selected from the group consisting of an enzyme, streptavidin, avidin, a fluorescent molecule, a luminescent molecule, a bioluminescent molecule, and a radioactive molecule.

In another embodiment, this document features an isolated cell that produces an antibody or fragment thereof, where the antibody or the fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide. The isolated cell can be a hybridoma. The antibody can be a monoclonal antibody.

In another embodiment, this document features a method for determining whether or not a sample contains a casp8p41 polypeptide. The method comprises or consists essentially of: a) contacting the sample with an antibody or fragment thereof to form a complex between the casp8p41 polypeptide, if present in the sample, and the antibody or the fragment thereof, where the antibody or the fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide; and b) determining the presence or absence of the complex, where the presence indicates that the sample contains the casp8p41 polypeptide, and where the absence indicates that the sample does not contain the casp8p41 polypeptide. The sample can be blood. The determining step can comprise or consist essentially of performing an ELISA or flow cytometry.

In another embodiment, this document features a method for determining whether or not a treatment for an immunodeficiency virus infection decreases cell death. The method comprises or consists essentially of determining whether or not the level of an IVP caspase polypeptide in a first sample from a mammal infected with an immunodeficiency virus is greater than or equal to the level of the IVP caspase polypeptide in a second sample from the mammal, where the first sample is from the mammal after the mammal received the treatment, where the second sample is obtained at a later time than the first sample, where a level of the IVP caspase polypeptide in the first sample that is equal to or greater than the level of the IVP caspase polypeptide in the second sample indicates that the treatment decreases cell death, and where a level of the IVP caspase polypeptide in the first sample that is lower than the level of the IVP caspase polypeptide in the second sample indicates that the treatment does not decrease cell death. The immunodeficiency virus can be a human immunodeficiency virus. The mammal can be a human. The IVP caspase polypeptide can be a casp8p41 polypeptide. The sample can be blood.

In another embodiment, this document features a method for monitoring an immunodeficiency virus infection. The method comprises or consists essentially of determining whether or not the level of an IVP caspase polypeptide in a first sample from a mammal infected with an immunodeficiency virus is greater than the level of the IVP caspase polypeptide in a second sample from the mammal, where the second sample is from the mammal at a time later than that of the first sample, where a level of the IVP caspase polypeptide in the first sample that is greater than the level of the IVP caspase polypeptide in the second sample indicates suppression of the infection, and where a level of the IVP caspase polypeptide in the first sample that is lower than the level of the IVP caspase polypeptide in the second sample indicates progression of the infection.

In another embodiment, this document features a method for determining the prognosis of an immunodeficiency virus infection. The method comprises or consists essentially of determining the level of an IVP caspase polypeptide in a mammal infected with an immunodeficiency virus, where a detectable level of the IVP caspase polypeptide indicates a poor prognosis, and where and an undetectable level of the IVP caspase polypeptide indicates a good prognosis. The determining step can comprise or consist essentially of analyzing a sample from the mammal.

In another embodiment, this document features a method for detecting the presence of an immunodeficiency virus in a mammal. The method comprises or consists essentially of determining whether or not an IVP caspase polypeptide is present within a mammal, where a detectable level of an IVP caspase polypeptide indicates the presence of an immunodeficiency virus, and where an undetectable level of the IVP caspase polypeptide indicates the absence of an immunodeficiency virus. The determining step can comprise or consist essentially of analyzing a sample from the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A is a graph plotting the proteolytic cleavage of a fluorogenic gag-pol substrate in Jurkat T cells that were either mock-infected (Mock) or infected with HIV IIIB. The inset is an immunoblot of cytosol and membrane fractions probed with anti-LAT antibodies.

FIG. 1B is a schematic diagram of a nucleic acid molecule encoding a procaspase-8 polypeptide with an amino-terminal Flag epitope and a carboxyl-terminal Myc epitope. Death effector domains (DEDs) are conserved amino acid sequences important for protein-protein interactions with other DED-containing proteins. P18 and P10 are further cleavage products of procaspase-8, which are assembled to create the catalytically active heterodimer, (P18/P10)₂. “FF” refers to the site where HIV protease cleaves, between two phenylalanine residues.

FIG. 1C (top panel on left) is an autoradiograph of a gel separating reaction mixtures containing ³⁵S methionine-labeled Flag-procaspase-8-Myc fusion polypeptide incubated in the presence (PR) or absence (CTR) of HIV-1 protease. FIG. 1C (middle panel on left) is an immunoblot of ³⁵S methionine-labeled Flag-procaspase-8-Myc fusion polypeptide incubated in the presence (PR) or absence (CTR) of HIV-1 protease and probed with an anti-Flag antibody. FIG. 1C (bottom panel on left) is an immunoblot of ³⁵S methionine-labeled Flag-procaspase-8-Myc fusion polypeptide incubated in the presence (PR) or absence (CTR) of HIV-1 protease and probed with an anti-Myc antibody. FIG. 1C, right panel contains an autoradiograph of a gel separating reaction mixtures containing ³⁵S methionine-labeled Flag-procaspase-8-Myc fusion polypeptide incubated in the presence of HIV-1 protease or renin, or untreated.

FIG. 1D is an autoradiograph of a gel separating reaction mixtures containing wild-type procaspase-8 polypeptides or procaspase-8 polypeptides with mutations F355R and F356N incubated with the indicated amounts of HIV protease.

FIG. 2A contains a series of immunoblots of cytosolic extracts from Jurkat T cells that were mock-treated (CTR), or incubated with recombinant HIV protease (PR) or GST alone (GST), recombinant GST casp8p41 (casp8p41) or campothecin (CPT). The cytosolic extracts were analyzed for Bid cleavage into p15 tBid, cytochrome c release into the cytosol, HSP70, and procaspase-9 cleavage.

FIG. 2B contains a series of photomicrographs of HeLa cells transfected with nucleic acid expressing GFP alone or GFPcasp8p41 and analyzed by confocal microscopy following DAPI and TUNEL staining. Camptothecin (CPT) treated cells and DNase I treated cells were included as controls.

FIGS. 2C and 2D contain histogram plots of data obtained from flow cytometric analyses of cells that were transfected with nucleic acid expressing GFP alone or GFPcasp8p41 and subjected to TUNEL staining (FIG. 2C, left panel), anti-active caspase-3 antibody staining (FIG. 2C, right panel), or Annexin-PE staining (FIG. 2D). The analyses were performed by gaiting on GFP-positive cells.

FIG. 2E contains a histogram plot and graph of data obtained from flow cytometric analyses of primary human T cells that were transfected with nucleic acid expressing GSK3β or casp8p41 and analyzed for changes in viability by light scatter or for apoptosis by TUNEL staining.

FIG. 2F contains a series of immunoblots of cytosolic extracts from Jurkat T cells that were mock-treated (CTR), or incubated with recombinant HIV protease (HIV-1PR) or recombinant casp8p41 polypeptide (casp8p41). The cytosolic extracts were analyzed for Bid cleavage into p15 tBid, cytochrome c release into the cytosol, HSP70, procaspase-9 cleavage, and PARP cleavage.

FIG. 3A contains immunoblots of extracts from cells transfected with nucleic acid expressing GFP, GFP full-length (FL) caspase-8, GFPcasp8p43, or GFPcasp8p41 that were probed with anti-casp8p41 or anti-GFP antibodies.

FIG. 3B contains a series of photomicrographs of HeLa cells transfected with nucleic acid expressing GFPcasp8p41, GFP FL caspase-8, or GFPcasp8p43 and analyzed by immunofluorescence with a casp8p41 monoclonal antibody or an isotype control antibody and PE-labeled secondary anti-mouse immunoglobulin antibodies.

FIG. 3C is a graph plotting casp8p41 production in Jurkat T cells that were transfected with nucleic acid expressing HIV protease and mock-treated (0), treated with the HIV-PR inhibitor Nelfinavir (Nfv), or treated with the pancaspase inhibitor Z-VAD fmk (Z-VAD).

FIG. 3D is a graph plotting the percentage of Jurkat T cells treated with the indicated stimuli that was positive for active caspase-3 (black bars) and casp8p41 (grey bars), as analyzed by flow cytometry. For each condition, the number of caspase-3+ cells present in the buffer or vehicle control treated cells was subtracted from the cells treated with the apoptosis inducing agents.

FIG. 4A contains a series of histograms plots of flow cytometric data from Jurkat T cells acutely infected with HIV IIIB that were analyzed for p24 and casp8p41 polypeptides on the indicated days after infection.

FIG. 4B is a graph plotting the percent of HIV infected Jurkat T cells that were casp8p41 polypeptide positive and Trypan blue positive on the indicated days after infection.

FIG. 4C contains histogram plots of flow cytometric data from Jurkat T cells acutely infected with HIV and analyzed for p41 polypeptide positivity and TUNEL staining 7 days after infection. Analyses were performed after gaiting on casp8p41 polypeptide negative cells (left panel) or casp8p41 polypeptide positive cells (right panel).

FIG. 5A is an immunoblot analyzing the expression of procaspase 8 in I9.2 cells that were mock-transfected, or stably transfected with either wild-type procaspase 8 (WT casp8) or procaspase 8 containing the F355R, F356N mutations (casp8 RN).

FIG. 5B is a graph plotting the percentage of viable I9.2 cells stably transfected with wild-type procaspase 8 (WT casp8) or procaspase 8 containing the F355R,F356N mutations (casp8 RN) and infected with VSV-G pseudotyped HIV. Viability was assessed as a function of time using Trypan blue.

FIG. 5C is a graph plotting the relative cell number of I9.2 cells stably transfected with wild-type procaspase 8 (WT casp8) or procaspase 8 containing the F355R,F356N mutations (casp8 RN) at 60 hours post-infection with VSV-G pseudotyped HIV. The relative cell number was measured based on ATP activity.

FIG. 5D is a graph plotting casp8p41 production in I9.2 cells stably transfected with wild-type procaspase 8 (WT casp8) or procaspase 8 containing the F355R,F356N mutations (casp8 RN) at 60 hours post-infection with VSV-G pseudotyped HIV. Results are representative of two independent experiments.

FIG. 6A is a graph plotting the percentage of casp8p41 positive cells in peripheral blood lymphocytes from HIV infected patients versus the viral load.

FIG. 6B contains a series of histogram plots of flow cytometric data analyzing peripheral blood lymphocytes from HIV-infected patients or uninfected donors for p24 polypeptide, casp8p41 polypeptide, and active caspase-3 polypeptide positivity.

FIG. 6C is a graph plotting the HIV viral load versus the percentage of p24 polypeptide positive cells that are positive for casp8p41 polypeptide.

FIG. 6D contains dot plots analyzing casp8p41, CD3, CD4, CD8, CD27, CD45RO, CD14, and CD19 expression in CD4⁺ T cells. Casp8p41 positive cells (red) were assessed for CD27 and CD45RO expression and compared to casp8p41 negative CD4 T cells.

FIG. 7A contains a series of photomicrographs of freshly frozen lymph nodes from HIV-negative donors or from HIV-infected patients being treated or not being treated with highly active antiretroviral therapy (HAART) that were analyzed by confocal microscopy after staining with anti-p24 polypeptide and anti-casp8p41 polypeptide antibodies and DAPI.

FIG. 7B contains a series of photomicrographs of lymph node sections similar to those in FIG. 7A that were analyzed for casp8p41 polypeptide, active caspase-3 polypeptide, and DAPI staining by confocal microscopy.

FIG. 8 is a listing of an amino acid sequence of a human caspase 8 polypeptide (SEQ ID NO:1).

FIG. 9 is a listing of an amino acid sequence of a human casp8p41 polypeptide (SEQ ID NO:2).

FIG. 10 is a listing of an amino acid sequence of a human caspase 10 polypeptide (SEQ ID NO:5).

FIG. 11 is a listing of an amino acid sequence of a human caspase 7 polypeptide (SEQ ID NO:6).

FIG. 12 is a listing of an amino acid sequence of a chimpanzee caspase 8 polypeptide (SEQ ID NO:7).

FIG. 13 is a listing of an amino acid sequence of a chimpanzee caspase 7 polypeptide (SEQ ID NO:8).

FIG. 14 is a listing of an amino acid sequence of a chimpanzee caspase 10 polypeptide (SEQ ID NO:9).

### DETAILED DESCRIPTION

This document provides methods and materials involved in identifying, monitoring, and prognosing immunodeficiency virus infections (e.g., HIV infections). For example, this document provides methods and materials for determining whether or not a mammal (e.g., a human) has an immunodeficiency virus infection (e.g., an HIV infection), methods and materials for determining whether or not a mammal with an immunodeficiency virus infection is improving, and methods and materials for determining whether or not a mammal with an immunodeficiency virus infection has an advanced immunodeficiency virus infection. This document also provides methods and materials for assessing the effectiveness of anti-immunodeficiency virus infection treatments. In addition, this document provides antibodies having the ability to bind to an IVP caspase polypeptide at a standard high affinity, while lacking the ability to bind to an uncleaved caspase polypeptide at that same affinity.

As described herein, this document provides methods and materials for identifying mammals having an immunodeficiency virus infection. Any mammal can be identified as having an immunodeficiency virus infection. For example, a human, a cat, or a monkey can be identified as having an immunodeficiency virus infection. In addition, any human can be identified as having an immunodeficiency virus infection. For example, an infant, a child, an adult, or a senior citizen of any race or gender can be diagnosed as having an immunodeficiency virus infection using the methods and materials provided herein.

An immunodeficiency virus can be any retrovirus causing immunosuppression, such as a human immunodeficiency virus (HIV), a feline immunodeficiency virus (FIV), or a simian immunodeficiency virus (SIV). In addition, an immunodeficiency virus can be any type of immunodeficiency virus, such as HIV type O, HIV type 1, or HIV type 2.

In some embodiments, a mammal can be identified as being infected with an immunodeficiency virus if it is determined that a sample (e.g., a blood or a lymph node sample) from the mammal contains an IVP caspase polypeptide.

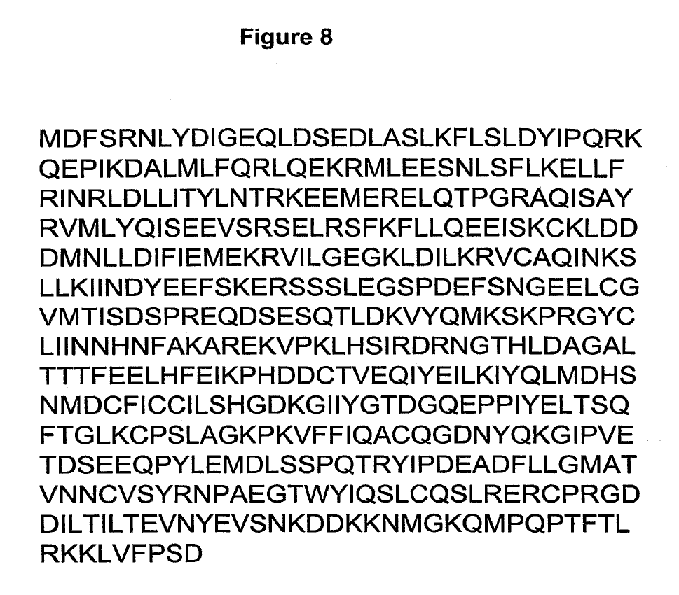

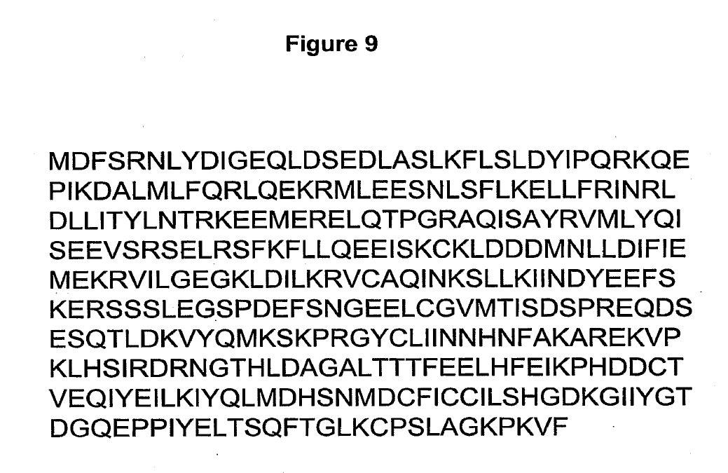

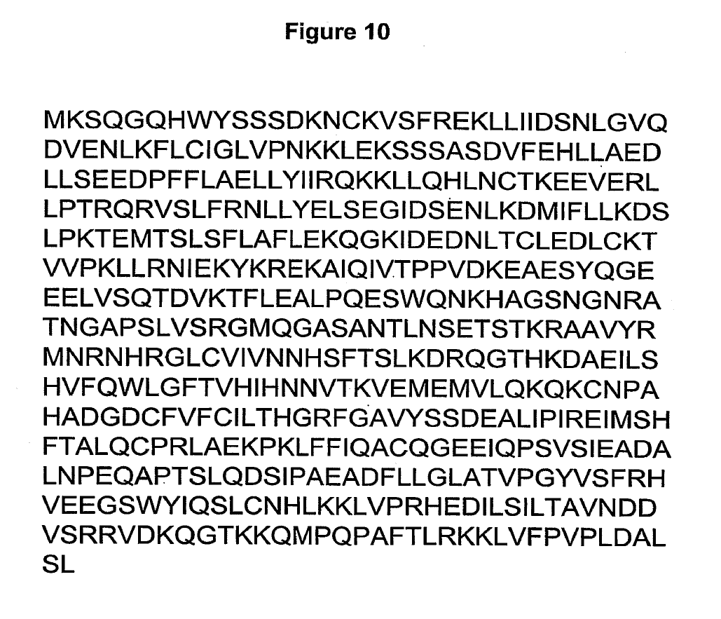

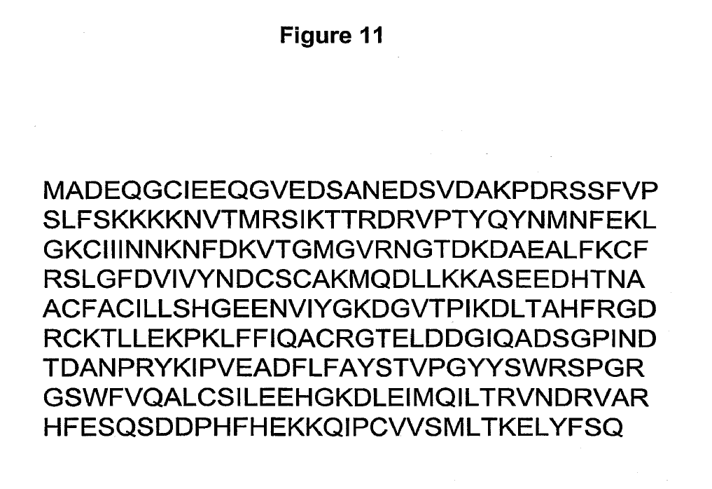

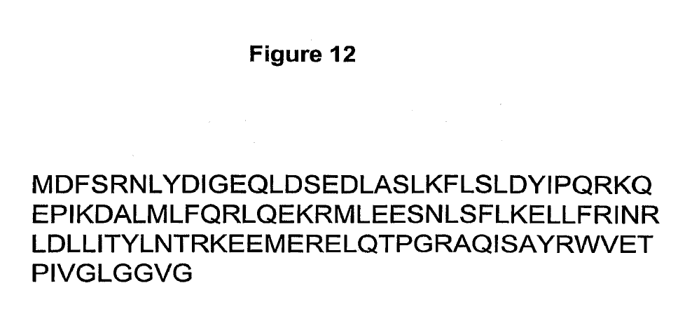

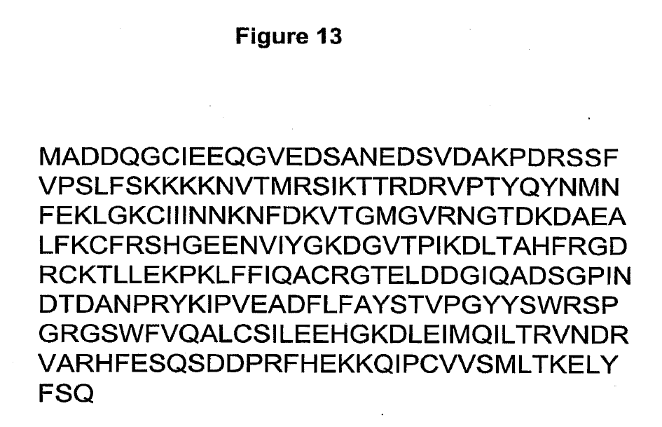

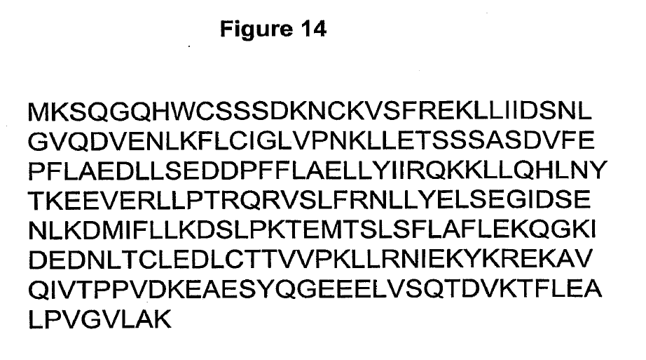

The term “IVP caspase polypeptide” as used herein refers to any polypeptide with the same amino acid sequence as a polypeptide fragment that can be formed from a cellular caspase polypeptide via cleavage by an immunodeficiency virus protease. For example, an IVP caspase polypeptide can be any polypeptide with the same amino acid sequence as a polypeptide fragment that formed from any cellular caspase polypeptide via cleavage by any immunodeficiency virus protease. Examples of cellular caspase polypeptides include, without limitation, human caspase 8 polypeptides, feline caspase 8 polypeptides, simian caspase 8 polypeptides, human caspase 10 polypeptides, feline caspase 10 polypeptides, simian caspase 10 polypeptides, human caspase 7 polypeptides, feline caspase 7 polypeptides, and simian caspase 7 polypeptides. The amino acid sequence of a human caspase 8 polypeptide can be as set forth in FIG. 8 and SEQ ID NO:1. The amino acid sequence of a simian caspase 8 polypeptide can be as set forth in FIG. 12 and SEQ ID NO:7. The amino acid sequence of a human caspase 10 polypeptide can be as set forth in FIG. 10 and SEQ ID NO:5. The amino acid sequence of a simian caspase 10 polypeptide can be as set forth in FIG. 14 and SEQ ID NO:9. The amino acid sequence of a human caspase 7 polypeptide can be as set forth in FIG. 11 and SEQ ID NO:6. The amino acid sequence of a simian caspase 7 polypeptide can be as set forth in FIG. 13 and SEQ ID NO:8. Examples of immunodeficiency virus proteases include, without limitation, HIV proteases, FIV proteases, and SIV proteases. An example of an IVP caspase polypeptide is a casp8p41 polypeptide, which can be generated via cleavage of a human procaspase-8 polypeptide by an HIV protease. The amino acid sequence of a casp8p41 polypeptide can be as set forth in FIG. 9 and SEQ ID NO:2. In some cases, an IVP caspase polypeptide can be a polypeptide generated via cleavage of a feline caspase polypeptide by an FIV protease. In some cases, an IVP caspase polypeptide can be a polypeptide generated via cleavage of a simian caspase polypeptide by an SW protease.

In some cases, a FLIP polypeptide fragment formed from a FLIP polypeptide via cleavage by an immunodeficiency virus protease can be used either (1) alone in place of an IVP caspase polypeptide, or (2) in combination with an IVP caspase polypeptide, to detect, monitor, and/or prognose immunodeficiency virus infections in mammals. An example of a nucleic acid encoding a human FLIP polypeptide is set forth in SEQ ID NO:15 and GenBank Accession No. NM_{—}003879. An amino acid sequence of a human FLIP polypeptide can be as set forth in SEQ ID NO:16 and GenBank Accession No. NM_{—}003879. In some cases, FLIP polypeptide fragments can be formed via cleavage of a FLIP polypeptide between amino acid residues corresponding to residues 355 and 356 of the amino acid sequence set forth in SEQ ID NO:16. A FLIP polypeptide fragment formed from a FLIP polypeptide via cleavage by an immunodeficiency virus protease can be used to generate anti-FLIP fragment antibodies. Such anti-FLIP fragment antibodies can be used to detect FLIP fragments in cells from a mammal. The presence of FLIP fragments (e.g., those FLIP fragments having a C-terminus corresponding to amino acid residue 355 of SEQ ID NO:16, or an N-terminus corresponding to amino acid residue 356 of SEQ ID NO:16) in a mammal can indicate that the mammal has an immunodeficiency virus infection.

Any method can be used to obtain a biological sample for evaluation. For example, blood can be obtained by peripheral venipuncture and evaluated for an IVP caspase polypeptide. In some cases, serum or plasma can be prepared from the blood and evaluated for an IVP caspase polypeptide. In some cases, lymphocytes can be isolated from peripheral blood using standard techniques, such as Ficoll-Hypaque density gradient separation, and evaluated for an IVP caspase polypeptide.

One or more samples can be collected from a mammal (e.g., a human) at one or more time points and evaluated for an IVP caspase polypeptide (e.g., a casp8p41 polypeptide). For example, a sample can be collected from an individual on a regular basis (e.g., every week, every two weeks, every month, every two months, every three months, every six months, or every year) and evaluated for a casp8p41 polypeptide. The frequency of sample collection and evaluation for an IVP caspase polypeptide also can vary over time. The levels of an IVP caspase polypeptide in multiple samples from the same mammal obtained at various times can be compared. Such a comparison can indicate, for example, the onset of an immunodeficiency virus infection or the progression of an immunodeficiency virus infection.

Once a biological sample, such as a blood or tissue sample, is obtained, the sample can be evaluated for an IVP caspase polypeptide. Alternatively, a biological sample can be divided into aliquots, preserved (e.g., fixed or frozen), and stored for analysis at a later time. For example, a sample can be frozen at a temperature of −20° C. or lower (e.g., −25° C., −75° C., −80° C., −100° C., or −200° C.) and stored frozen for days, months, or years. A frozen sample can be thawed and evaluated for an IVP caspase polypeptide. In some cases, a biological sample, such as a tissue specimen, can be formaldehyde-fixed and paraffin-embedded, sectioned, and stored at room temperature for days, months, or years prior to being evaluated for an IVP caspase polypeptide.

Any method can be used to determine the level of an IVP caspase polypeptide in a biological sample. For example, the level of an IVP caspase polypeptide in a biological sample can be determined using, without limitation, ELISA, radioimmunoassay, fluoroimmunoassay, Western blotting, flow cytometry, immunohistochemistry, immunoprecipitation, confocal microscopy, or mass spectrometry.

In some embodiments, the methods and materials provided herein can be used to identify mammals as being infected with an immunodeficiency virus in cases where serologic assays can be unreliable. For example, the methods and materials provided herein can be used at a stage before seroconversion to identify a mammal as being infected with an immunodeficiency virus. In some cases, the methods and materials provided herein can be used to identify a mammal having an acute immunodeficiency virus infection. The methods and materials provided herein also can be used to identify a child infected with an immunodeficiency virus through the route of maternal fetal transmission.

This document also provides methods and materials for monitoring the course of an immunodeficiency virus infection (e.g., an HIV infection) in a mammal (e.g., a human). For example, the course of an HIV infection can be monitored by comparing the level of a casp8p41 polypeptide in a sample from an individual to the level of a casp8p41 polypeptide in a second sample obtained from the same individual at an earlier point in time. In some cases, a measurable increase in the amount of a casp8p41 polypeptide in a sample from an individual relative to the amount of a casp8p41 polypeptide in a second sample from the same individual taken at an earlier time point (e.g., one week, two weeks, a month, two months, six months, one year, two years, or five years earlier) can indicate progression of the HIV infection. In some cases, a measurable decrease in the amount of a casp8p41 polypeptide in a sample from an individual relative to the amount of a casp8p41 polypeptide in a second sample from the same individual taken at an earlier time point (e.g., one week, two weeks, a month, two months, six months, one year, two years, or five years earlier) can indicate suppression of the HIV infection. For example, a measurable decrease in the amount of a casp8p41 polypeptide in a sample from an individual relative to the amount of a casp8p41 polypeptide in a second sample from the same individual taken at an earlier time point (e.g., one week, two weeks, a month, two months, six months, one year, two years, or five years earlier) can indicate reduced HIV replication.

Samples collected from the same mammal (e.g., human) at different time points can be preserved and stored, as described above, so that they can be evaluated simultaneously. Alternatively, the samples can be analyzed individually, e.g., at their respective times of collection, and the IVP caspase polypeptide levels of samples analyzed at later time points can be compared to the IVP caspase polypeptide levels of samples analyzed at earlier time points.

Samples also can be divided into aliquots once they are collected, and an aliquot of each sample can be analyzed at each time point for an IVP caspase polypeptide.

The course of an immunodeficiency virus infection in a mammal can be monitored on a regular basis (e.g., every week, every two weeks, every month, every two months, every three months, every six months, or every year) or on an irregular basis over the course of weeks, months, years, or a lifetime. In addition, the course of an immunodeficiency virus infection can be monitored in any mammal infected with an immunodeficiency virus. For example, the course of an HIV infection can be monitored in an HIV infected infant, child, adult, or senior citizen of any race or gender.

This document also provides methods and materials for assessing the effectiveness of an immunodeficiency virus (e.g., HIV) treatment, such as highly active antiretroviral therapy (HAART), in a mammal (e.g., a human) infected with an immunodeficiency virus. For example, the effectiveness of an immunodeficiency virus treatment can be assessed by comparing the level of an IVP caspase polypeptide (e.g., a casp8p41 polypeptide) in one sample from a mammal having received the treatment to the level of an IVP caspase polypeptide in another sample obtained from the same mammal at an earlier time point. In some cases, a measurable decrease in the amount of an IVP caspase polypeptide in a sample from a mammal having received an immunodeficiency virus treatment relative to the amount of an IVP caspase polypeptide in an earlier obtained sample from the same mammal (e.g., a sample obtained one day, two days, three days, four days, five days, six days, seven days, eight days, nine days, ten days, 11 days, 12 days, 13 days, 14 days, 15 days, 16 days, 17 days, 18 days, 19 days, 20 days, 21 days, one month, two months, six months, one year, two years, five years, seven years, or ten years earlier) can indicate that the immunodeficiency virus treatment is effective. In some cases, a measurable increase in the amount of an IVP caspase polypeptide in a sample from a mammal having received an immunodeficiency virus treatment relative to the amount of an IVP caspase polypeptide in an earlier obtained sample from the same mammal (e.g., a sample obtained one day, two days, three days, four days, five days, six days, seven days, eight days, nine days, ten days, 11 days, 12 days, 13 days, 14 days, 15 days, 16 days, 17 days, 18 days, 19 days, 20 days, 21 days, one month, two months, six months, one year, two years, five years, seven years, or ten years earlier) can indicate that the immunodeficiency virus therapy is not effective. The earlier obtained sample collected from the mammal can be collected during or prior to treatment of the mammal with an immunodeficiency virus therapy. For example, the earlier obtained sample can be collected one, two, three, four, five, six, seven, eight, nine, 10, 11, 12, 13, 14, 15, 16, 17, 18, 19, 20, 21, or more days before treatment of the mammal with an immunodeficiency virus therapy. The sample collected subsequent to collection of the earlier obtained sample can be collected during or following treatment of the mammal having an immunodeficiency virus infection.

Any number of samples collected from a mammal having an immunodeficiency virus infection at two or more time points can be evaluated for IVP caspase polypeptide levels to monitor the effectiveness of an immunodeficiency virus therapy. For example, samples obtained from a mammal prior to receiving an immunodeficiency virus therapy and once a week for one year during treatment can be used to monitor the effectiveness of an immunodeficiency virus treatment. Comparing the levels of an IVP caspase polypeptide in the samples can indicate whether or not the therapy is effective. For example, a decrease in the level of an PVP caspase polypeptide over time can indicate that the therapy is effective. In some cases, a lack of a decrease (e.g., an increase) in the level of an IVP caspase polypeptide over time can indicate that the therapy is not effective. In some cases, a decrease in the level of an IVP caspase polypeptide over an initial time period followed by an increase in the level of an IVP caspase polypeptide can indicate that the therapy is no longer effective, possibly due to viral resistance to the therapy.

The effectiveness of an immunodeficiency virus (e.g., HIV) therapy can be assessed in any mammal (e.g., human) having an immunodeficiency virus infection and having received an immunodeficiency virus therapy. For example, the effectiveness of an HIV therapy can be assessed in an infant, a child, an adult, or a senior citizen of any race or gender having an HIV infection and having received an HIV therapy.

This document also provides methods for determining prognosis in immunodeficiency virus (e.g., HIV) infection. For example, the prognosis of a mammal (e.g., a human) infected with an immunodeficiency virus (e.g., HIV) can be determined by determining the level of an IVP caspase polypeptide (e.g., a casp8p41 polypeptide) in a sample (e.g., a sample comprising CD4⁺ cells) from the mammal. In some cases, the presence of an IVP caspase polypeptide in a sample from a mammal can indicate a rapid depletion of CD4⁺ cells and, therefore, a poor prognosis. In some cases, the absence of a detectable level of an IVP caspase polypeptide in a sample from a mammal can indicate a slow depletion of CD4⁺ cells and, therefore, a good prognosis.

In some cases, the level of an IVP caspase polypeptide can be measured in addition to the level of another polypeptide, and the ratio of the levels can be used to determine a mammal's prognosis. For example, a ratio of an IVP caspase polypeptide to an immunodeficiency virus p24 polypeptide can be used to determine a mammal's prognosis. Any method can be used to determine the level of an immunodeficiency virus (e.g., HIV) p24 polypeptide in a biological sample. For example, an ELISA (Beckman Coulter, Brea, Calif.) can be used to measure the level of an HIV p24 polypeptide in serum or plasma.

The prognosis of a mammal infected with an immunodeficiency virus also can be determined by measuring the level of an IVP caspase polypeptide in a sample from the mammal and determining the mammal's CD4⁺ cell count. For example, an undetectable level of an IVP caspase polypeptide coupled with a CD4⁺ cell count greater than 500 CD4⁺ cells/mm³, e.g., greater than 550, 600, 650, 700, 750, 800, 850, 900, 950, 1000, 1050, 1100, 1150, 1200, 1250, 1300, 1350, 1400, 1450, 1500, 1550; or 1600 CD4⁺ cells/mm³, can indicate a good prognosis. Determining the prognosis of a mammal infected with an immunodeficiency virus can allow clinicians to design the most appropriate therapeutic strategies. For example, an HIV patient having a poor prognosis may warrant a more aggressive treatment strategy and vice versa.

Any method can be used to determine a mammal's CD4⁺ cell count. For example, absolute CD4⁺ cell counts can be determined based on immunophenotypic identification of cells with fluorescently labeled monoclonal antibodies directed against the CD4 antigen. Relative percentages of CD4⁺ T cells can be determined with a flow cytometer. An absolute CD4⁺ cell count can be derived by multiplying the percentage of lymphocytes that are CD3⁺/CD4⁺ by the absolute lymphocyte count determined with a hematology instrument. Alternatively, TruCount Absolute Count Tubes (BD Biosciences, San Jose, Calif.) can be used to measure CD4⁺ cell levels in HIV-infected patients.

This document also provides antibodies that bind to an IVP caspase polypeptide. Such antibodies can have the ability to bind to an IVP caspase polypeptide at an affinity of at least 10⁴ mol⁻¹, e.g., at least 10⁵, 10⁶, 10⁷, 10⁸, 10⁹, 10¹⁰, 10¹¹, or 10¹² mol⁻¹, while lacking the ability to bind to an uncleaved version of the cellular IVP caspase polypeptide corresponding to the IVP caspase polypeptide at that affinity. For example, an antibody provided herein can have the ability to bind to human casp8p41 at an affinity of at least 10⁴ mol⁻¹, e.g., at least 10⁵, 10⁶, 10⁷, 10⁸, 10⁹, 10¹⁰, 10¹¹, 10¹² mol⁻¹, while lacking the ability to bind to an uncleaved human caspase 8 polypeptide at that affinity. In some cases, the antibody can bind to an IVP caspase polypeptide at an affinity between 10⁴ to 10¹³ mol⁻¹, 10⁴ to 10¹² mol⁻¹, 10⁴ to 10¹¹ mol⁻¹, 10⁴ to 10¹⁰ mol⁻¹, 10⁵ to 10¹³ mol⁻¹, 10⁵ to 10¹² mol⁻¹, 10⁵ to 10¹¹ mol⁻¹, 10⁵ to 10¹⁰ mol⁻¹, 10⁶ to 10¹³ mol⁻¹, 10⁶ to 10¹² mol⁻¹, 10⁶ to 10¹¹ mol⁻¹, or 10⁶ to 10¹⁰ mol⁻¹. In some cases, the antibody can bind to an IVP caspase polypeptide at a concentration of 0.05 to 1.0 μg/mL (e.g., 0.1 to 0.5 μg/mL, 0.1 to 0.4 μg/mL, or 0.08 to 0.8 μg/mL) without observable cross-reactivity to an uncleaved version of the IVP caspase polypeptide via flow cytometry or immunofluorescence. In some cases, an antibody provided herein can bind to a casp8p41 polypeptide (e.g., SEQ ID NO:2), a polypeptide corresponding to the carboxy terminal end of a casp8p41 polypeptide (e.g., SEQ ID NO:3), or a polypeptide comprising the four carboxy terminal amino acids of a casp8p41 polypeptide (e.g., SEQ ID NO:4), as determined by immunoassays well known in the art for assaying antibody-antigen binding.

The term “antibody” as used herein includes, without limitation, polyclonal, monoclonal, multi-specific, human, humanized, chimeric, and single-chain antibodies as well as antibody fragments having binding activity such as Fab fragments, F(ab') fragments, F(ab')2 fragments, Fd fragments, fragments produced by a Fab expression library, fragments comprising a VL or VH domain, and epitope binding fragments of any of the above. An antibody can be of any type (e.g., IgG, IgE, IgM, IgD, IgA, and IgY), class (e.g., IgG1, IgG2, IgG3, IgG4, IgA1, and IgA2), or subclass. In addition, antibodies can be from any animal including birds and mammals. For example, antibodies can be human, murine, rabbit, goat, guinea pig, camel, horse, or chicken.

An antibody can be naturally occurring, recombinant, or synthetic. Antibodies can be generated by any suitable method known in the art. For example, monoclonal antibodies can be prepared using a wide variety of techniques known in the art, including the use of hybridoma, recombinant, and phage display technologies, or a combination thereof. An antibody such as an antibody fragment can be produced by any means. For example, an antibody fragment can be enzymatically or chemically produced by fragmentation of an intact antibody. An antibody fragment can also be produced synthetically or recombinantly from a gene encoding the partial antibody sequence.

Once an antibody has been produced by an animal, chemically synthesized, or recombinantly expressed, it can be purified by any method known in the art. For example, an antibody can be purified by chromatography, centrifugation, or differential solubility. In some cases, an antibody can be fused to a sequence, such as a hexa-histidine peptide, for convenient purification of the fusion protein. A substantially pure preparation of an antibody can be enriched for the antibody. For example, a substantially pure antibody preparation can be enriched such that 20, 30, 40, 50, 60, 70, 80, 90, or 100% of the antibodies present in the preparation are identical.

Antibodies provided herein can be used, without limitation, to purify or detect an IVP caspase polypeptide (e.g., a casp8p41 polypeptide). For example, the antibodies can be used in immunoassays for qualitatively and quantitatively measuring levels of casp8p41 polypeptides in biological samples (e.g., blood samples). The antibodies can be recombinantly fused or conjugated to molecules useful as labels in detection assays, such as enzymes, streptavidin, avidin, fluorescent molecules, luminescent molecules, bioluminescent molecules, and radioactive molecules. A detectable substance can be coupled or conjugated either directly to the antibody or fragment thereof, or indirectly, e.g., through a linker. In some cases, an antibody can be fused to another molecule (e.g., a polypeptide) to aid in the uptake of the antibody into cells. See, e.g., Mie et al., Biochem. Biophys. Res. Comm., 310(3): 730-734 (2003)).

The invention will be further described in the following examples, which do not limit the scope of the invention described in the claims.

### EXAMPLES

### Example 1

### Detecting HIV Protease Activity in HIV-Infected Cells

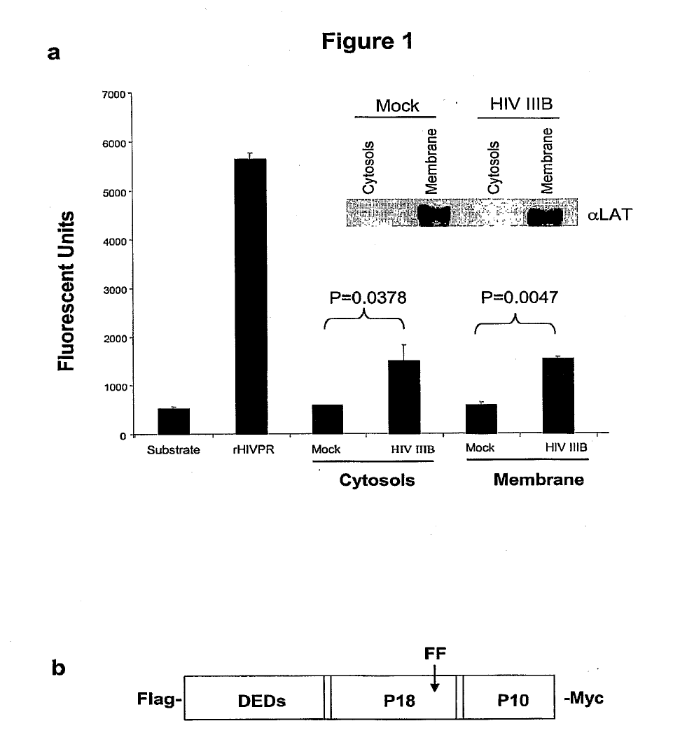

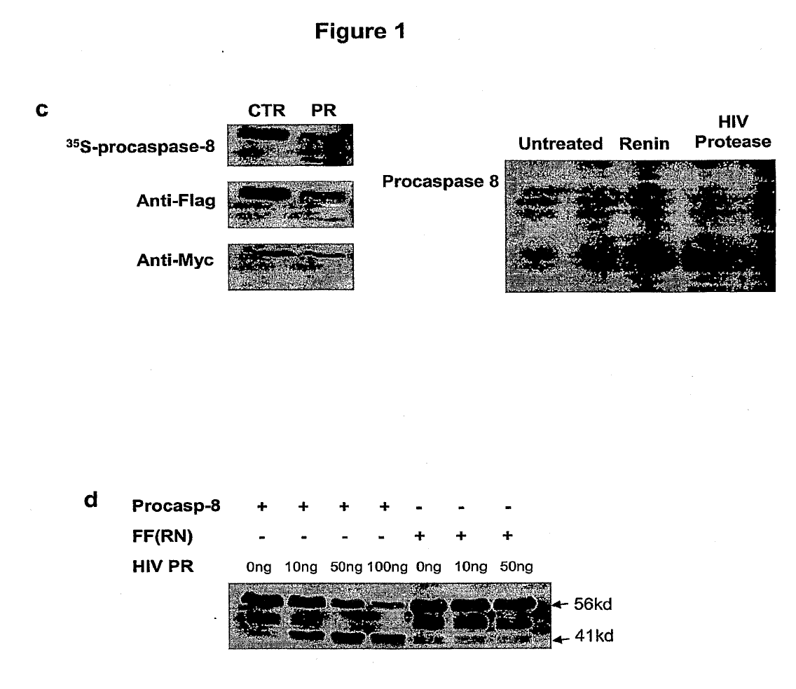

HIV protease activity was assessed during HIV infection to determine whether the protease was active in the cytosolic compartment and the membrane fraction. Jurkat T cells were infected with HIV, the cytosolic and membrane fractions were separated, and the purity was confirmed by analysis of the membrane-specific marker LAT using immunoblotting (FIG. 1A, inset; Tridandapani et al., J Biol Chem, 275:20480-20487 (2000)). Each fraction was assessed for protease activity using a fluorogenic gag-pol substrate.

To directly measure the protease activity in the cytoplasmic compartment of the infected cells, 200×10⁶ HIV-infected Jurkat cells were harvested when the infected cells started to die (typically at day three or four post-infection). The harvested cells were washed twice to remove the cell surface attached virus particles, and were then resuspended in swelling buffer (10 mM Tris-HCl, pH 7.4, 5 mM MgC12, 30 mM NaCl, 1 mM EGTA, 1 mM DTT, 100 mM PMSF, 10 μg/mL leupeptin, 2 μg/mL aprotinin). After incubation on ice for 15 minutes, the cells were disrupted with 30 strokes of a tight-fitting “B” type glass Dounce homogenizer. The nuclei and subcellular organelles were removed by centrifugation at 10,000 rpm for 30 minutes at 4° C. The supernatant from this spin was further centrifuged for one hour at 100,000×g using a Beckman Rotor SW Ti55 with 2 mL Quick-Seal centrifuge tubes. The resulting supernatant (S100) was collected as cytosolic fraction and the pellet, membrane fraction, was suspended with 500 μL of the swelling buffer supplemented with 0.1% Triton×100. The same numbers of uninfected Jurkat cells were processed in parallel according to the same protocol. The protease activities were measured using equivalent amounts of proteins from both the cytosolic fractions (S-100) and the membrane fractions using a fluorogenic substrate, as we described elsewhere (Cuerrier et al., Biochem Biophys Res Commun., 327:208-211 (2005)).

The membrane fraction of HIV-infected cells contained more protease activity than the membrane fraction of uninfected cells (P=0.0047; FIG. 1A).

Moreover, the cytosolic fraction of HIV-infected cells contained significantly more protease activity than uninfected cells (P=0.0378; FIG. 1A). These results indicate that HIV protease is active in the cytoplasm of infected T cells. These results also indicate that protease cleavage of cytosolic host cell proteins may contribute to viral cytopathicity.

### Example 2

### Identifying the Site of Cleavage of Procaspase-8 by HIV Protease

To identify the site of cleavage of procaspase-8 by HIV protease, a procaspase-8 expression construct was constructed with an amino terminal Flag epitope and a carboxyl terminal Myc epitope (FIG. 1B). The following primers were used to amplify a Flag-procaspase-8-Myc fragment from a pcDNA3procaspase8 plasmid using the polymerase chain reaction (PCR): 5′CCCAAGCTTATGGACTACAAAGACGATGACGGTACCATGGACTTCAG C-3′ (Flag_sense; SEQ ID NO:10), and 5′-CCGGGCCCTTACTACAGATCCTCTTCTGAGATGAGTTTTTGTTCTCTAGATTGATCAGAAGGGAAAA G-3′ (Myc_antisense; SEQ ID NO:11). The Flag-procaspase-8-Myc fragment from the PCR was inserted back into HindIII and Xbal sites of the pcDNA3 vector, and the resulting construct was designated pcDNA3Flag-procaspase-8-Myc.

The fusion polypeptide, Flag-procaspase-8-Myc, was prepared by TNTR Quick coupled transcription/translation systems (Promega, Madison, Wis.). Briefly, 1 μg of pcDNA3Flag-procaspase-8-Myc or pcDNA3Flag (as a control) was added to a 50 μL nuclease-free reaction mixture containing 40 μL of TNT Quick Master Mix and 1 μL of 1 mM ³⁵S methionine. The reaction was carried out at 30° C. for 90 minutes and stopped by adding 20 μg/mL of cycloheximide (CI-IX) and 200 μg/mL of RNase and incubating the mixture at 30° C. for 5 minutes. ³⁵S methionine-labeled Flag-procaspase-8-Myc fusion polypeptide was prepared by adding 40 μCi of ³⁵S methionine instead of 1 μL of 1 mM methionine. The reaction mixture was separated by SDS-PAGE on a 10-15% gel and visualized by autoradiography. IVTT products were cleaned using a G50 Column (Amersham Biosciences, Piscataway, N.J.), and reconstituted in caspase buffer (25 mM HEPES, pH 7.5, 0.15 M NaCl, 5 mM DTT, 10% sucrose).

The ³⁵S methionine-labeled Flag-procaspase-8-Myc fusion polypeptide produced in vitro was incubated with HIV-1 protease purchased from Sachem Biosciences (King of Prussia, Pa.). The HIV-1 protease had a specific activity of 1.81×10⁴ mM/min./mg at 37° C. and a purity of greater than 96 percent by SDS-PAGE. RP-HPLC analysis of the HIV-1 protease produced a single peak. HIV-1 protease assays were performed in a buffer containing 50 mM NaOAc (pH 4.9), 200 mM NaCl, 5 mM DTT, and 10% glycerol. After cleavage of ³⁵S methionine-labeled Flag-procaspase-8-Myc fusion polypeptide by HIV-1 protease, the products were analyzed by autoradiography (FIG. 1C, top) and immunoblotting using antibodies directed against the Flag (FIG. 1C, middle) and Myc (FIG. 1C, bottom) epitopes. HIV-protease cleavage of ³⁵S methionine-labeled Flag-procaspase-8-Myc fusion polypeptide resulted in a predominant band containing a Flag epitope. These results suggested that the cleavage site was located within the carboxyl terminal half of the p18 subunit of procaspase-8. In contrast, another aspartyl protease, renin, did not cause procaspase 8 cleavage (FIG. 1C, right panel).

Highly purified procaspase-8 p18 subunit was cleaved with HIV protease, and the resultant cleavage products were sequenced by mass spectroscopy following tryptic digestion. The recombinant active caspase-8 was purchased from R & D Systems (Minneapolis, Minn.). After cleavage by HIV-1 protease, the products were separated by SDS-PAGE and stained using silver stain plus (Bio-Rad, Hercules, Calif.). The silver stained bands were digested with trypsin, and the extracted peptides were analyzed by LC-tandem mass spectrometry.

A Waters Micromass Q-T of API-US quadrupole time-of-flight mass spectrometer with a Z-spray ESI source was coupled to a CapLC HPLC system (Waters, Milford, Mass.). Both instruments were controlled by MassLynx 4.0. HPLC was carried out by running a gradient of increasing acetonitrile in 0.1% formic acid on a Vydac C4 column (300 μm×100 mm) at a flow rate of 4 μL/min., and eluting directly into the mass spectrometer. The mass spectrometry experiment consisted of continuous 2 second scans of 400-2000 m/z, and the spectra were deconvoluted using the MassLynx MaxEnt algorithm to give monoisotopic masses.

Tryptic digestion of the procaspase-8 p18 subunit generated three specific peptides: RGYCLIINNHNFAK (N-terminal; SEQ ID NO:12), KGIIYGTDGQEAPIYELTSQFTGLK (SEQ ID NO:13), and KVFFIQACQGDNYQK (C-terminal; SEQ ID NO:14). When the procaspase-8 p18 subunit was digested with HIV protease prior to digesting with trypsin, the tryptic peptide KVFFIQACQGDNYQK (C-terminal; SEQ ID NO:14) was lost.

These results indicated that the cleavage site for HIV protease resides within the C-terminal tryptic fragment. To define the precise cleavage site for HIV protease, the molecular weight of the cleaved p18 fragment was measured by LC-MS. The sample of procaspase-8 p18 cleaved by HIV protease had a fragment that was not present in the sample of procaspase-8 p18 that was not cleaved by HIV protease. This fragment had a weight of 16158 mass units (cleaved form), indicating that procaspase-8 p18 was cleaved by HIV protease between Phe355 and Phe356.

To confirm this result, amino acids 355 and 356 were mutated in full-length caspase-8 to reduce the likelihood of HIV protease cleavage at that site. Mutations were selected based on a Markov chain model of HIV protease substrate cleavage (Chou, Anal Biochem., 233:1-14 (1996); Chou and Zhang, J Protein Chem., 12:709-724 (1993)). The wild-type octapeptide surrounding amino acids 355/356 was predicted to be cleaved by HIV protease with high efficiency, whereas the F355R, F356N mutation was predicted to be cleaved much less efficiently. Phe355 and Phe356 were replaced with Arg355 and Asn356, respectively, in plasmid pcDNA3Flag-procaspase-8-Myc using the QuikChange® site-directed mutagenesis kit (Stratagene, La Jolla, Calif.). ³⁵S-methionine labeled wild-type procaspase-8 and the mutant procaspase-8 F355R, F356N were reacted with HIV protease and analyzed for cleavage by autoradiography. Incubation of wild-type procaspase-8 with HIV protease resulted in a p41 procaspase-8 fragment. In contrast, an almost negligible amount of cleavage was observed when procaspase-8 F355R, F356N was incubated with HIV protease (FIG. 1D).

### Example 3

### Determining that casp8p41 Polypeptide Causes Apoptosis

The ability of the procaspase-8 cleavage fragment p41 (hereafter referred to as casp8p41) to initiate an apoptotic signaling cascade in a cell-free system was tested. The cell free system was used previously to reveal the signaling pathway used by HIV protease to initiate apoptosis.

Jurkat cells were purchased from ATCC (Rockville, Md.) and maintained in Dulbecco's Modified Eagle Medium (DMEM) or RPMI1640 (GIBCO) supplemented with 10% (v/v) FBS, 1% penicillin and streptomycin, and 2 mM L-glutamine. The cells were incubated at 37° C. in a humidified atmosphere containing 5% CO₂ and 95% air. Jurkat cell extracts were prepared as described previously (Nie et al., Cell Death Differ., 9:1172-84 (2002)). Briefly, cells (0.5×10⁶ cells/mL) were harvested by centrifugation at 16000×g for 5 minutes at 4° C. The cell pellet was washed twice with ice-cold PBS (pH 7.4), followed by a single wash with ice-cold caspase buffer (20 mM PIPES, 100 mM NaCl, 10 mM DTT, 1 mM EDTA, 0.1% CHAPS, 250 mM sucrose, pH 7.2). After centrifugation, the cells were resuspended in two volumes of ice-cold complete caspase buffer, which was supplemented with protease inhibitors (100 mM PMSF, 20 μM leupeptin, and 0.3 μM aprotinin). The cells were then transferred to a 2-mL Dounce homogenizer. After incubating on ice for 15 minutes, the cells were disrupted with 50 strokes using a B-type pestle (Fisher Scientific Ltd., Nepean, ON, Canada). Cell disruption (>95%) was confirmed by examining a 5 μL aliquot of the suspension under a light microscope after staining with Trypan blue. The nuclei were removed by centrifugation at 1000×g for 10 minutes at 4° C. Polypeptide concentrations were determined using a BCA protein assay kit (Pierce Chemical Co., Rockford, Ill.), and the resultant suspension was incubated with either HIV protease or casp8p41 polypeptide.

For Western blot analysis, 50-200 μg of cytosolic polypeptides were fractionated on 10% or 15% polyacryamide gels and transferred onto PVDF membranes (Millipore, Bedford, Mass.) for 3 hours at 100 V in transfer buffer (25 mM Tris, 192 mM glycine). The membranes were blocked by incubating them overnight at 4° C. or 2 hours at room temperature in TBS buffer (20 mM Tris, 150 mM NaCl, 0.05% Tween, pH 7.5) containing 2% BSA. The membranes were then washed five times with TBS buffer and blotted for 1 hour at room temperature with the following primary antibodies: monoclonal anti-caspase-8 (Biosource International, Camarillo, Calif.), anti-caspase-9 (Medical & Biological Laboratories Co., Watertown, Mass.), goat anti-BID (Santa Cruz Biotechnology), anti-cytochrome c and anti-PARP (BD Pharmingen, Mississauga, ON, Canada). The blots were washed five times with TBS and developed with HRP-linked secondary antibodies: sheep anti-mouse IgG, donkey anti-rabbit Ig (Amersham Pharmacia, Oakville, ON, Canada), and anti-goat IgG (Santa Cruz Biotechnology). All of the blots were developed using SuperSignal (Pierce, Rockford, Ill.), an enhanced chemiluminescence method, according to the manufacturer's protocol.

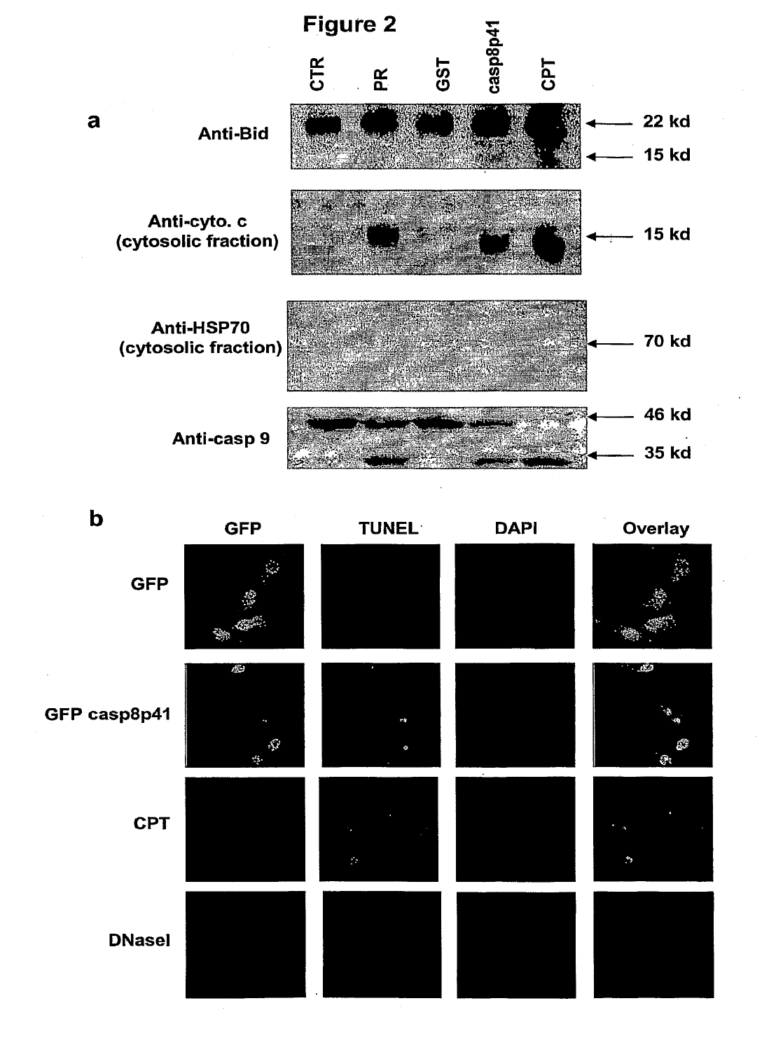

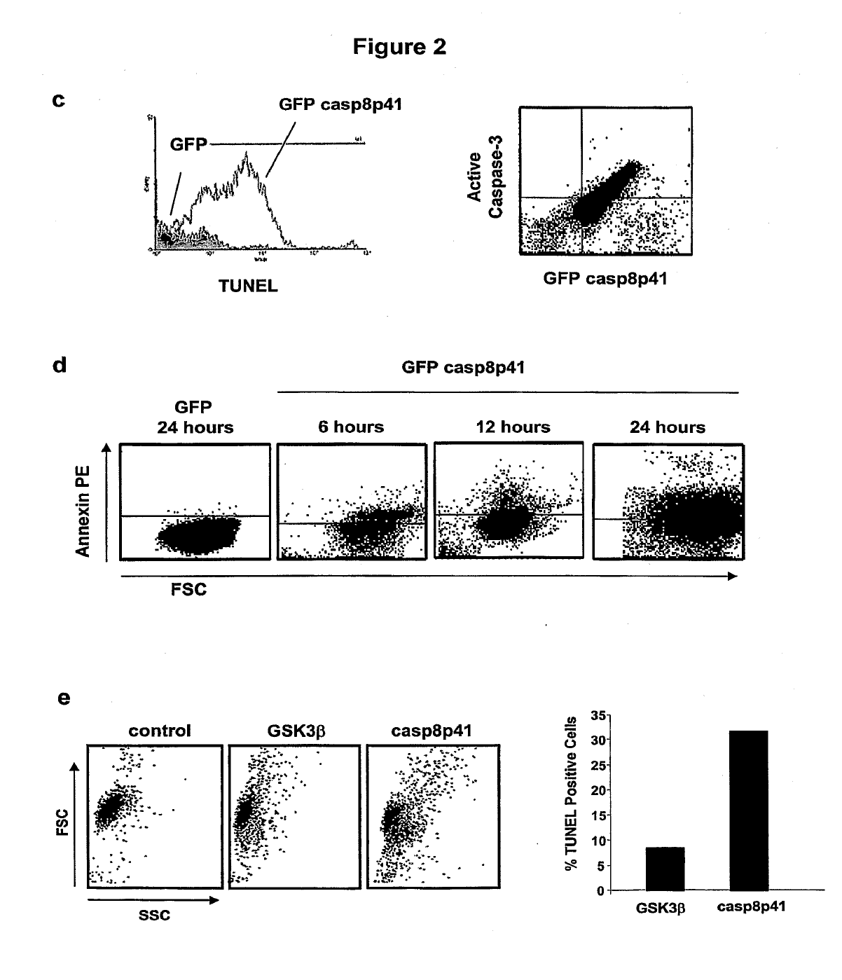

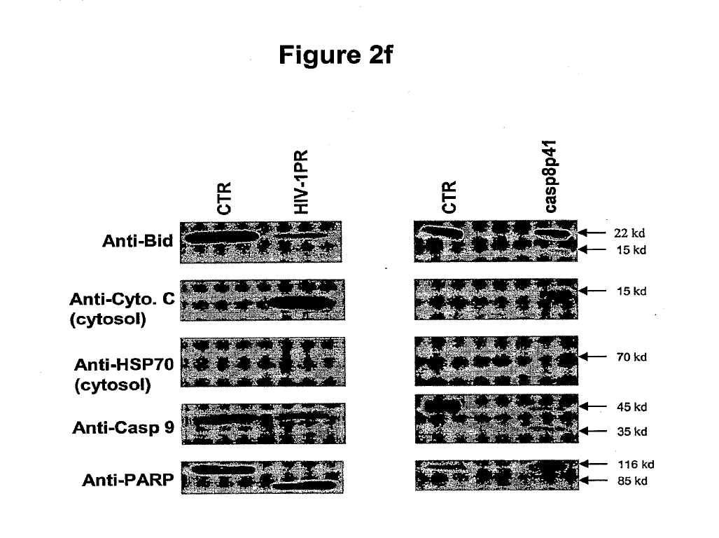

Adding either casp8p41 polypeptide or HIV protease to cytosols from Jurkat T cells resulted in cleavage of Bid to p15 tBid, release of cytochrome c from the mitochondrial fraction into the cytosol, and cleavage of caspase-9 and PARP (FIGS. 2A and 2F). The effects produced by casp8p41 polypeptide and HIV protease were indistinguishable. These findings indicated that casp8p41 polypeptide is able to initiate apoptosis in a manner similar to HIV protease.

The effect of transient expression of casp8p41 polypeptide in HeLa cells was investigated. HeLa cells were purchased from ATCC and maintained in Dulbecco's Modified Eagle Medium (DMEM; GIBCO) supplemented with 10% (v/v) FBS, 1% penicillin and streptomycin, and 2 mM L-glutamine. The cells were incubated at 37° C. in a humidified atmosphere containing 5% CO₂ and 95% air. Exponentially growing cells were seeded at 2500-5000 cells/well in a 96-well plate. The cells were transfected with a DNA plasmid expressing GFP (pEGFPC1, BD Biosciences Clontech, Palo Alto, Calif.), or with a plasmid expressing casp8p41 polypeptide as a fusion to the C-terminus of GFP (pEGFPC1casp8p41) using Fugene 6 (2 μL per μg DNA), according to the manufacturer's protocol. The pEGFPC1casp8p41 plasmid was constructed by inserting the gene sequence for the full length procaspase-8 protein into the BamHI site of pEGFPC1 and then using site directed mutagenesis to change the Phe at 356 to a stop codon. The plasmids were purified by centrifugation on a CsCl gradient prior to transfection.

HeLa cells transfected with pEGFPC1 or pEGFPC1casp8p41 were analyzed by confocal microscopy following staining with DAPI to visualize cell nuclei and TUNEL staining to detect apoptosis. Cells treated with camptothecin or DNase I were included as controls. Transient expression of GFPcasp8p41 in HeLa cells caused TUNEL-positive apoptosis of GFP-positive cells, but not GFP-negative cells. GFP-positive cells transfected with the GFP control vector were also negative for TUNEL (FIG. 2B). These observations indicated that casp8p41 polypeptide initiated apoptosis in cells in which it was expressed, but did not cause apoptosis of bystander cells.

Cells transfected with pEGFPC1 and pEGFPC1casp8p41 were also subjected to Annexin-V PE, active caspase-3, and TUNEL staining and analyzed for cell death by flow cytometry, gating specifically on GFP-positive cells. To perform the annexin-V staining assay, 1×10⁶ cells were harvested, washed, and stained with 2 μL annexin-V PE (BD Pharmingen, San Diego, Calif.) at 37° C. for 20 minutes. GFP and annexin-V PE double-positive cells were analyzed by flow cytometry at 30,000 events per sample. TUNEL staining for detection of apoptosis was performed according to the manufacturer's protocol (Roche, Nutley, N.J.). Flow cytometric analyses indicated that casp8p41 polypeptide induces TUNEL positivity and active caspase-3 expression (FIG. 2C) in a time-dependent manner (FIG. 2D).

Primary human T cells were tested for their response to expression of casp8p41 polypeptide by transfecting cells with a vector encoding casp8p41 polypeptide, or with a control vector encoding glycogen synthase kinase 3β (GSK3β; Kim et al., J Biol Chem., 278:13995-14001 (2003)). PBL from HIV-negative donors were isolated and transfected with 2 μg/10×10⁶ cells with pcDNA3 HA GSK3β (Ougolkov et al., Cancer Res., 65:2076-2081 (2005)) or pcDNA3 HA casp8p41 using nucleofectin (AMAXA, MD) and program U14. Transfected cells were analyzed for changes in viability by light scatter or for apoptosis by TUNEL staining.

While transfection of primary T cells with the GSK3β vector induced detectable apoptosis, transfection with the vector encoding casp8p41 polypeptide induced a significantly greater level of apoptosis (FIG. 2E). These results confirmed that casp8p41 polypeptide is proapoptotic in T cells, and suggested that it is a potent death stimulus.

### Example 4

### Developing an Anti-casp8p41 Antibody

During death receptor-initiated apoptosis, procaspase-8 is cleaved through induced proximity autoactivation (Boatright et al., Mol. Cell, 11:529-41 (2003)). This process generates a p43 fragment of procaspase-8 by cleavage after amino acid Asp374 (Thornberry and Molineaux, Protein Sci., 4:3-12 (1995)). The process whereby the p43 fragment of procaspase-8 is generated is distinct from the cleavage event that generates casp8p41 polypeptide. Since subsequent processing of caspase-8 is not known to induce cleavage at residues F355 and F356, the neoepitope which is generated at the carboxyl terminus of the casp8p41 fragment should not be generated during caspase-8 processing that does not involve HIV protease. To investigate this possibility, the carboxyl terminus of the casp8p41 polypeptide was used as an immunogen to generate a monoclonal antibody directed against the casp8p41 fragment.

A peptide corresponding to the C-terminal end of the caspase-8 p41 polypeptide (CPSLAGKPKVF; SEQ ID NO:3) was synthesized and linked to KLH via the N-terminal cysteine. An anti-casp8p41 monoclonal antibody was produced as described elsewhere (de StGroth and Scheidegger, J. Immunol. Methods, 35:1-21 (1980)). Initial screening was done by ELISA using the full-length antigen peptide. Subsequent screening and selection of hybridomas was completed using a peptide consisting of a seven alanine leader and the four C-terminal amino acids of the antigen peptide (AAAAAAAPKVF; SEQ ID NO:4). The selected clones were then screened by immunofluorescence and Western blotting to ensure specificity for the p41 fragment of caspase-8.

HeLa cells were transfected with a plasmid expressing GFP or with a plasmid expressing full-length caspase-8 polypeptide, casp8p43 polypeptide, or casp8p41 polypeptide as a fusion to the C-terminus of GFP (designated GFP FLcasp8, GFPcasp8p43, and GFPcasp8p41, respectively). To generate the GFP FLcasp8 plasmid, the full coding sequence of the human procaspase-8 gene was cloned into the BamHI site of pEGFPC1 (BD Biosciences Clontech). The GFPcasp8p43 and GFPcasp8p41 constructs were created by inserting a stop codon at amino acid 375 or 356, respectively, using the QuikChange® site-directed mutagenesis kit (Stratagene).

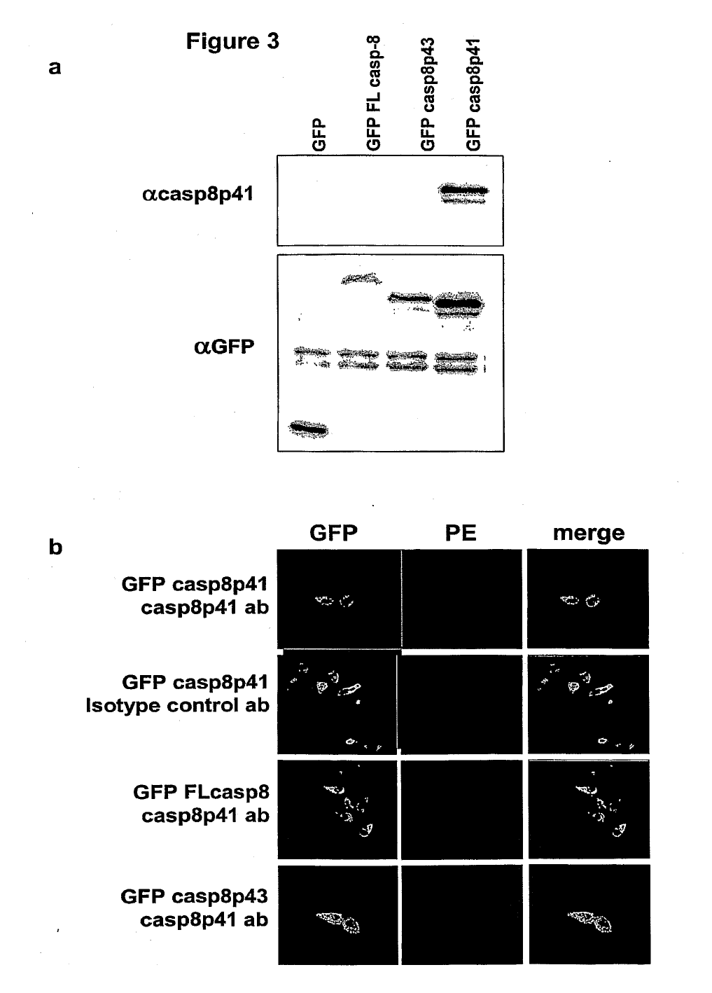

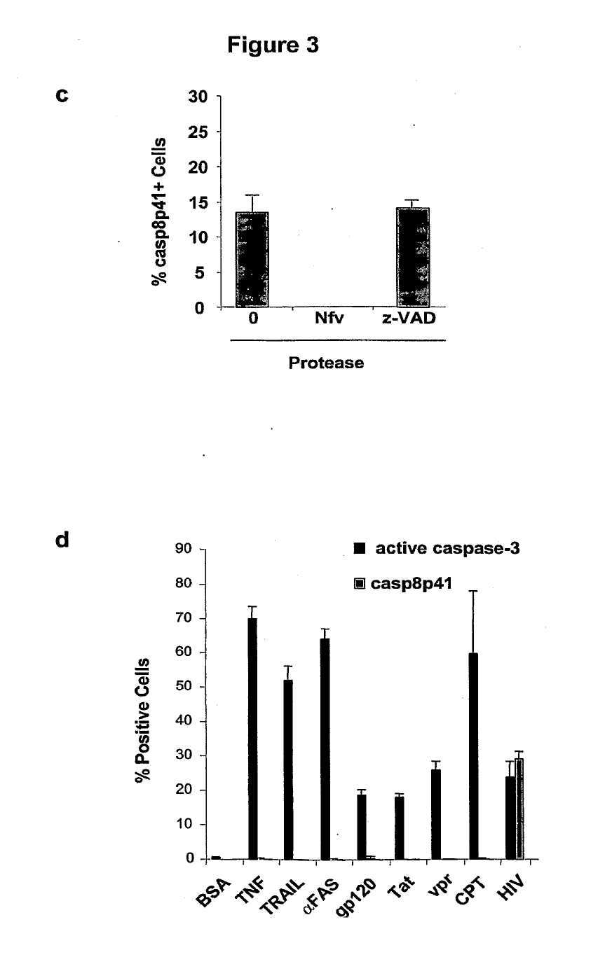

Lysates from the transfected cells were analyzed by immunoblotting using anti-casp8p41 or anti-GFP antibodies. Only cells transfected with GFPcasp8p41 contained polypeptide that was recognized by the casp8p41 antibody, whereas all cells contained polypeptide that was detected by the anti-GFP antibody (FIG. 3A). HeLa cells transfected with GFPcasp8p41, GFP FLcasp8, or GFPcasp8p43 were also analyzed by immunofluorescence using either the casp8p41 monoclonal antibody or an isotype control antibody along with PE-conjugated secondary anti-mouse immunoglobulin antibodies. Only those cells transfected with a plasmid expressing casp8p41 polypeptide and stained with the casp8p41 antibody were PE positive (FIG. 3B). These results indicated that the anti-casp8p41 monoclonal antibody recognized casp8p41 polypeptide, but not casp8p43 polypeptide or full-length procaspase-8 polypeptide, by immunoblotting (FIG. 3A) as well as immunohistochemistry (FIG. 3B).

### Example 5

### Analyzing casp8p41 Polypeptide in HIV Infected Cells In Vitro

To determine whether casp8p41 is produced specifically as a consequence of HIV-PR activity, as opposed to being produced as a consequence of apoptotic processing of procaspase 8, casp8p41 expression was assessed following HIV-PR transfection in the presence or absence of HIV-PR inhibitor, Nelfinavir, or the pan-caspase inhibitor, z-VAD-fmk. Jurkat T cells were transfected with 1 μg of pEYFP-protease per 10⁷ cells using a square wave electroporator (BTX) at 320 V for 10 msec. Immediately following transfection, 7 μM Nelfinavir or 20 μM Z-VAD-fmk was added. The following morning, casp8p41 was assessed by flow cytometry. Casp8p41 was detectable in the HIV-PR transfected cells, and inhibiting protease activity abrogated casp8p41 production (FIG. 3C). In contrast, pan-caspase inhibition did not alter casp8p41 production, indicating that HIV protease, but not caspase processing, is required for casp8p41 production.

It was determined whether casp8p41 is generated in response to other apoptotic stimuli. Jurkat T cells (10⁶) were treated with 10 μM campothecin, 100 ng/mL SuperKillerTRAIL™ (Axxora), 250 ng/mL anti-Fas antibody CH-11 (Upstate Cell Signaling Solutions), 50 ng/mL TNF-α (R&D Systems) plus 5 μg/mL cycloheximide, 2 μM HIV Tat (NIH AIDS Research and Reference Reagent Program), 1 μg/mL of HIV gp120 (ImmunoDiagnostic), or vehicle controls for 8 hours at 37° C. For Vpr-induced apoptosis, Jurkat T cells were incubated with isotonic glucose-Hepes buffer (2.4% glucose, 13 mM Hepes, 68 mM NaCl, 1.3 mM KCl, 4 mM Na2HPO4, and 0.7 mM KH2PO4, pH 7.2) alone or containing 10 μM of Vpr-derived peptide (amino acids 61-75; NIH AIDS Research and Reference Reagent Program) for 4 hours at 37° C. After the incubation, cells were washed in PBS and incubated overnight at 37° C. After incubation for the indicated times, cells were fixed in 2% paraformaldehyde at 4° C. overnight. To determine the presence of casp8p41 on apoptotic cells, cells were permeabilized with PBS+0.1% NP-40 on ice for two minutes. Cells were then incubated with the mouse anti-casp8p41 antibody, followed by PE-labeled goat anti-mouse antibody (Becton Dickinson ImmunoCytochemistry) and FITC-labeled rabbit anti-active caspase-3 antibody.

Treatment of cells with TNF-α, TRAIL, and Fas ligation, stimuli that induce caspase-8 dependent apoptosis, did indeed induce apoptosis, as determined by activation of caspase-3. However, these stimuli did not generate casp8p41 immunoreactivity (FIG. 3D). The HIV proteins gp120, Tat, and Vpr induce apoptosis and have been implicated in HIV-induced T-cell death.

Stimulation of Jurkat cells with these proteins resulted in active caspase-3 staining, but casp8p41 was not detected. Likewise, casp8p41 was not produced by induction of apoptosis using campothecin. To test whether casp8p41 could be detected in cells experimentally infected with HIV, HIV-infected cells were analyzed for casp8p41 and active caspase 3 three days following infection. Approximately 25% of cells were apoptotic (active caspase-3+), and casp8p41 was detected in a similar percentage of cells. Thus, casp8p41 is a selective marker of HIV protease-induced apoptosis that is not generated by death-receptor signaling, or by other stimuli that have been implicated in HIV-associated T cell death. Further, since casp8p41 is readily detectable in experimental HIV infection, this stimulus may be relevant to killing of infected cells.

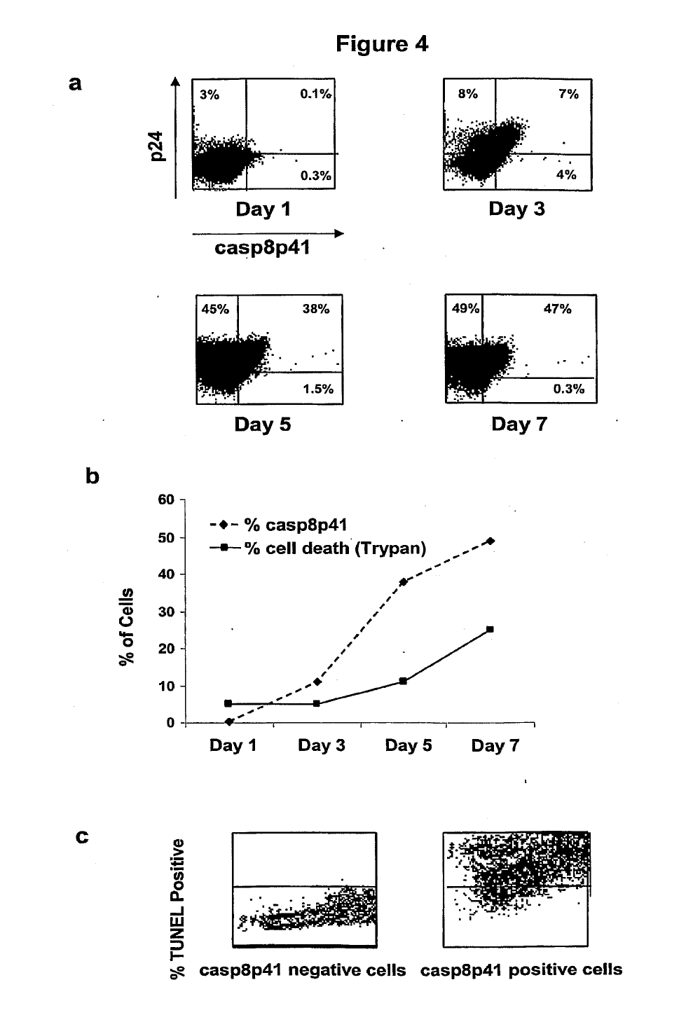

To determine whether casp8p41 polypeptide is generated during infection with HIV, Jurkat T cells acutely infected with HIV TUB were analyzed by flow cytometry for p24 polypeptide and casp8p41 polypeptide. Casp8p41 immunoreactivity was observed in Jurkat T cells infected with HIV IIIb, but not in mock-infected control cells, indicating that HIV protease-mediated cleavage of procaspase-8 had occurred (FIG. 4A). Importantly, casp8p41 staining was observed exclusively in cells coexpressing p24, which is also a product of HIV protease-mediated processing. Thus, casp8p41 reactivity was only detected in cells having evidence of protease activity and direct HIV infection. In these infections, casp8p41 immunoreactivity varied directly with both the proportion of cells expressing p24 (FIG. 4A) and with cell death (FIG. 4B). These results suggested that infection, death, and the presence of casp8p41 polypeptide were linked.

This association was directly assessed by analyzing the proportion of cells positive for casp8p41 polypeptide that was apoptotic. If casp8p41 polypeptide were not responsible for death of infected cells, then the proportion of dying cells expressing casp8p41 polypeptide would be expected to be similar to the proportion of viable cells expressing casp8p41 polypeptide. Jurkat T cells acutely infected with HIV were analyzed on day 7 after infection for casp8p41 positivity and for apoptosis using the TUNEL assay. In these cultures, fewer than 10% of the casp8p41 polypeptide negative cells were TUNEL positive, whereas 92% of casp8p41 polypeptide positive cells were apoptotic (FIG. 4C).

Together with the data indicating that casp8p41 polypeptide can induce apoptotic signaling in a cell-free system, and the data demonstrating that expression of casp8p41 polypeptide alone can initiate apoptosis, these findings demonstrated that casp8p41 polypeptide is a cause of death of cells infected with HIV in vitro.

### Example 6

### Analyzing casp8p41 Production in HIV-Induced Cell Death

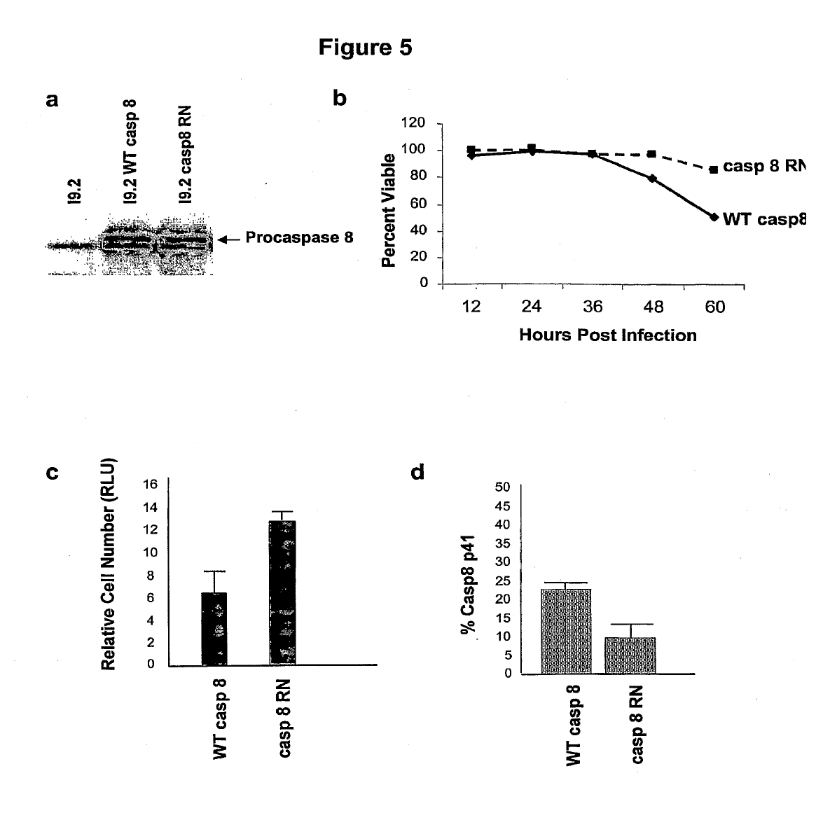

To assess the relative contribution of HIV-PR cleavage of procaspase 8 to the overall killing of T cells acutely infected with HIV, caspase-8 deficient I9.2 cells were stably transfected with a vector construct encoding full-length wild-type caspase 8 (casp8 WT), or full-length caspase 8 with the F355R, F356N mutations (casp8 RN; FIG. 5A). Since these cells have a low level of CD4 expression, robust HIV infection is not possible. Therefore, these cells were infected with VSV-G-pseudotyped HW instead. VSV-G infections were performed using an MO' of 1.0, as described elsewhere (Lum et al., J Clin Invest., 111:1547-1554 (2003)). Infected cells were analyzed for viability (FIG. 5B), cell number (FIG. 5C), and casp8p41 expression (FIG. 5D). Cell viability was assessed using a cell titer glow luminescent assay (Promega, Madison, Wis.), according to the manufacturer's instructions. Using this assay, viability is determined based on quantitation of ATP as a marker of metabolically active cells.

Infection of I9.2 cells transfected with the vector construct encoding wild-type caspase 8 resulted in high levels of death in cells expressing casp8p41. Infection of I9.2 cells transfected with the vector construct encoding caspase 8 with F355R, F356N mutations resulted in less casp8p41 production and less VSV-G HIV-induced death. These results are in agreement with the observation that the casp8 F355R, F356N mutant is less susceptible to cleavage by HIV protease (FIG. 1D). Thus, introduction of a mutation in procaspase-8, which reduces the amount of casp8p41 polypeptide produced, reduces the amount of HIV-induced cell death.

### Example 7

### Analyzing casp8p41 Polypeptide in Cells from HIV Infected Patients

The association between viral replication, casp8p41 polypeptide expression, and apoptosis was assessed in cells from HIV-infected patients. Peripheral blood lymphocytes (PBLs) from HIV-infected patients or HIV-negative controls were collected following informed consent, harvested using Ficoll hypaque, and analyzed for p24 and casp8p41 positivity. PBLs (1×10⁶ cells) were fixed with 2% paraformaldehyde in PBS and then permeabilized by incubation in PBS with 0.1% NP40 on ice for 2 minutes. Casp8p41 antibodies were added to the permeabilized cells, followed by addition of PE-labeled secondary antibodies (Becton Dickinson Immunocytometry Systems, San Jose, Calif.) and then anti-p24-FITC antibodies. Flow cytometry was performed using a FACScan (Becton Dickinson Immunocytometry Systems), and analysis was done using CellQuest software.

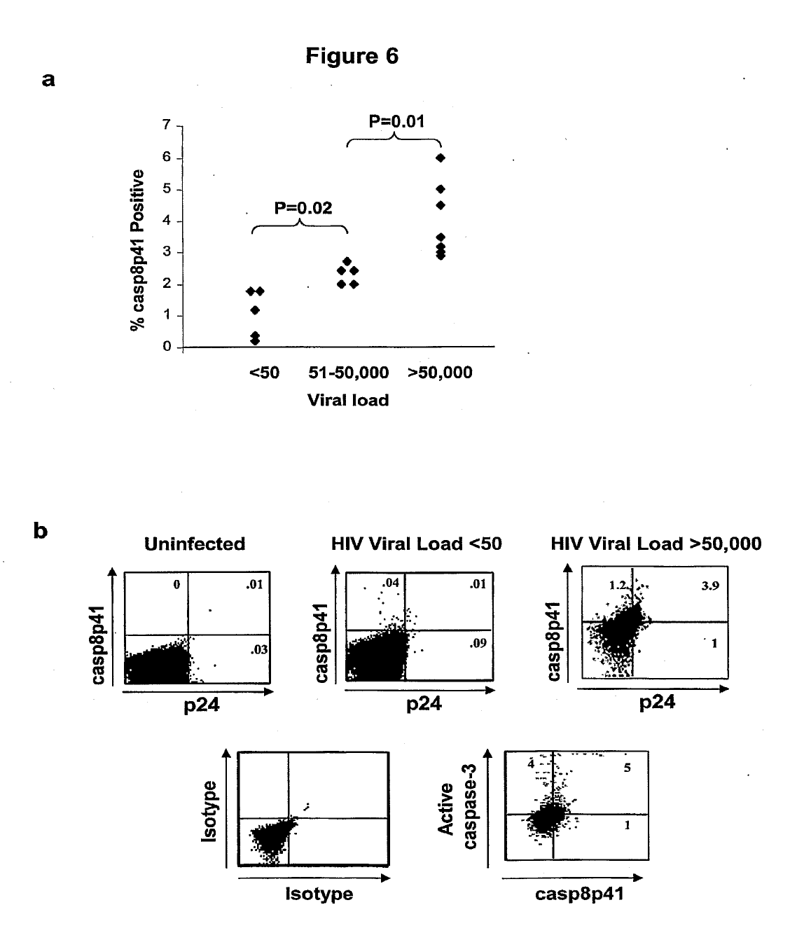

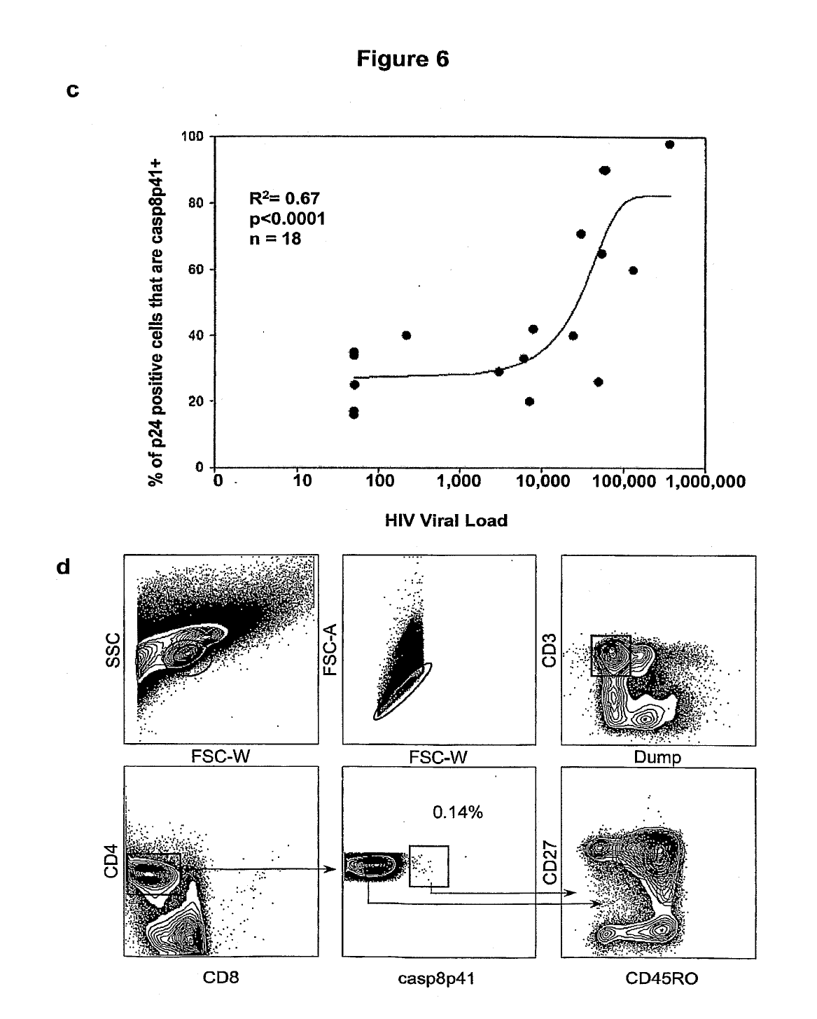

A small proportion of cells are physically infected with HIV. Consistent with this, a minority of cells were casp8p41 positive in bulk PBL populations. In patients with suppressed levels of viral replication, less than 2% of cells contained casp8p41. In patients with greater levels of viral replication, greater levels of casp8p41 were detected (FIG. 6A). In cells co-stained for p24 as a marker of cellular infection, the majority of p24 positive were also casp8p41 positive, and the majority of casp8p41 positive cells were p24 positive (FIG. 6B, top). Cells from HIV-negative patients did not contain p24 or casp8p41 polypeptides. Cells from patients with <50 copies of plasma viremia had far fewer casp8p41+ cells than cells from patients with active viral replication, indicating both that reducing viral replication reduces production of casp8p41, and that undetectable viremia is not equivalent to absent replication (FIG. 6B, top). Consistent with the cell-free data, as well as the transfection and in vitro infection data described herein, cells containing casp8p41 were apoptotic as determined by active caspase-3 staining (FIG. 6B, bottom).

Both in vitro and in vivo, some casp8p41+ cells did not stain for p24. It is possible that this occurs when intracellular infection is insufficient to generate detectable p24, but does produce detectable casp8p41. This possibility was assessed by analyzing the proportion of p24+ cells that contain casp8p41 in patients with varying degrees of viral replication. In patients with undetectable viral replication, rare p24+ cells were observed, demonstrating that suppressive antiviral therapy does not totally eliminate intracellular replication. Casp8p41 polypeptide was present in a third of those p24+ cells (FIG. 6C). However, as viral replication increased, the proportion of p24+ cells containing casp8p41 increased. In some cases, virtually all p24+ cells contained casp8p41. Logistic regression confirmed the striking direct association between viral replication and the proportion of p24+ cells containing casp8p41.

The involvement of casp8p41 polypeptide in killing of cells in lymphatic tissues from patients infected with HIV was evaluated. Freshly frozen tissues were stained with DAPI, anti-casp8p41-Alexaflour 555 antibodies, and anti-p24-FITC or anti-active caspase-3 antibodies. Laser-scanning confocal microscopy was performed using a Zeiss LSM-510 laser scanning confocal microscope (Carl Zeiss Inc., Thornwood, N.Y.). Images were saved at eight bits per channel and analyzed for fluorescent interaction using the computer analysis package ANALYZE (Mayo Foundation, Rochester, Minn.).

Polychromatic flow cytometry was used to determine how casp8p41 expression is localized within individual T cell subsets in vivo. Peripheral blood mononuclear cells (PBMC) from HIV-infected and uninfected individuals were stained with monoclonal antibodies against CD3, CD4, CD8, CD45RO, CD27, p41, CD14, and CD16. Monoclonal antibodies used for phenotypic characterization of T-cell subsets were anti-CD19 Cascade Blue, anti-CD14 Cascade Blue, anti-CD27 PE, anti-p41 Alexa 647, anti-CD45RO FITC (Coulter), CD3 Cy7APC, CD8 QDot705, CD4 Cy5.5PE and (Caltag). Unconjugated antibodies against CD19, CD14, and CD8 were obtained from BD Pharmingen, and p41 antibody was produced, purified and then conjugated with the Alexa 647 (Invitrogen) using standard protocols (described at drmr.cotn/abcon on the World Wide Web). Qdot conjugations were performed as described elsewhere (Chattopadhyay et al., Nature Medicine (2006)). All sorts were performed on stained cells fixed with 1% paraformaldehyde (PFA; Electron Microscopy Sciences, Ft. Washington, Pa.) using a modified FACS Aria (BD). Instrument set up was performed according to manufacturer's instructions. All sorts were performed at 25 PSI. Instrument compensation was performed using antibody-capture beads (BD Pharmingen) stained singly with individual antibodies used in the test samples.

A representative dot plot is presented in FIG. 6D. The highest frequency of casp8p41+ cells belonged to the CD27+ memory CD4+ T cell subset (Table 1), consistent with other studies demonstrating that CD27+ memory CD4+ T cells represent the most frequently infected T cell subset in vivo (Brenchley et al., J. Virol., 78:1160-1168 (2004)). The frequency of casp8p41+ cells in HIV-uninfected individuals was negligible. Further, small numbers of casp8p41+ memory CD4+ T cells could be sorted from all HIV+ individuals. Quantitative PCR was performed to analyze viral DNA, based on the gating scheme presented in FIG. 6D. An ABI7700 (Perkin-Elmer, Norwalk, Conn.) instrument was used to quantify HIV DNA, as described elsewhere (Douek et al., Nature, 417:95-98 (2002)). To quantify cell number in each reaction, quantitative PCR was performed simultaneously for albumin gene copy number as described elsewhere (Douek et al., Nature, 417:95-98 (2002)). Standards were constructed for absolute quantification of gag and albumin copy number, and were validated with sequential dilutions of 8E5 and Ach2 cell lysates containing one copy of gag per cell. Duplicate reactions were analyzed and template copies calculated using ABI7700 software. In all cases, viral DNA was present in casp8p41+ memory CD4+ T cells (Table 1).

TABLE 1
Frequency of casp8p41 in CD4+ T cell subsets and CD8+ T cells.

| | | | | | | % of |
|---|---|---|---|---|---|---|
| | | | % | | | memory |
| | | | memory | % naive | | CD4 that |
| | | | CD4 | CD4 | % CD8 | contain |
| Subject | CD4 | VL | casp8p41+ | casp8p41+ | casp8p41+ | viral DNA |
| 1 | 552 | 22,000 | 0.06 | 0.01 | ≦0.009 | 0.1 |
| 2 | 145 | 35,000 | 0.09 | 0.03 | ≦0.009 | 6.1 |
| 3 | 205 | 54,000 | 0.1 | 0.04 | ≦0.009 | 0.51 |
| 4 | 752 | 18,000 | 0.07 | 0.02 | ≦0.009 | 0.97 |
| 5 | HIV− | | ≦0.009 | ≦0.009 | ≦0.009 | |
| 6 | HIV− | | ≦0.009 | ≦0.009 | ≦0.009 | |
| 7 | HIV− | | ≦0.009 | ≦0.009 | ≦0.009 | |
| 8 | HIV− | | ≦0.009 | ≦0.009 | ≦0.009 | |

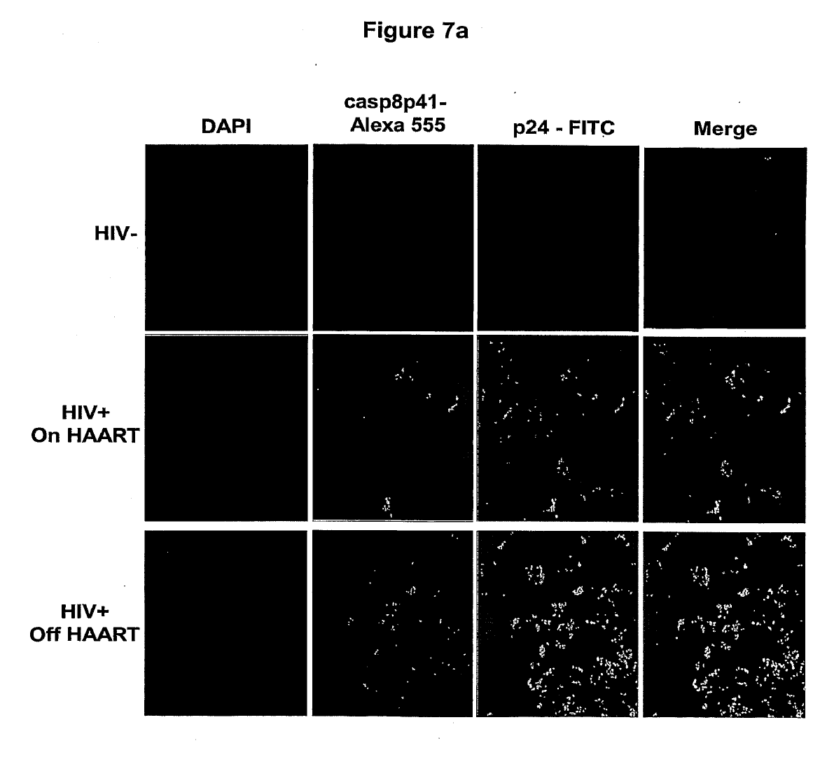

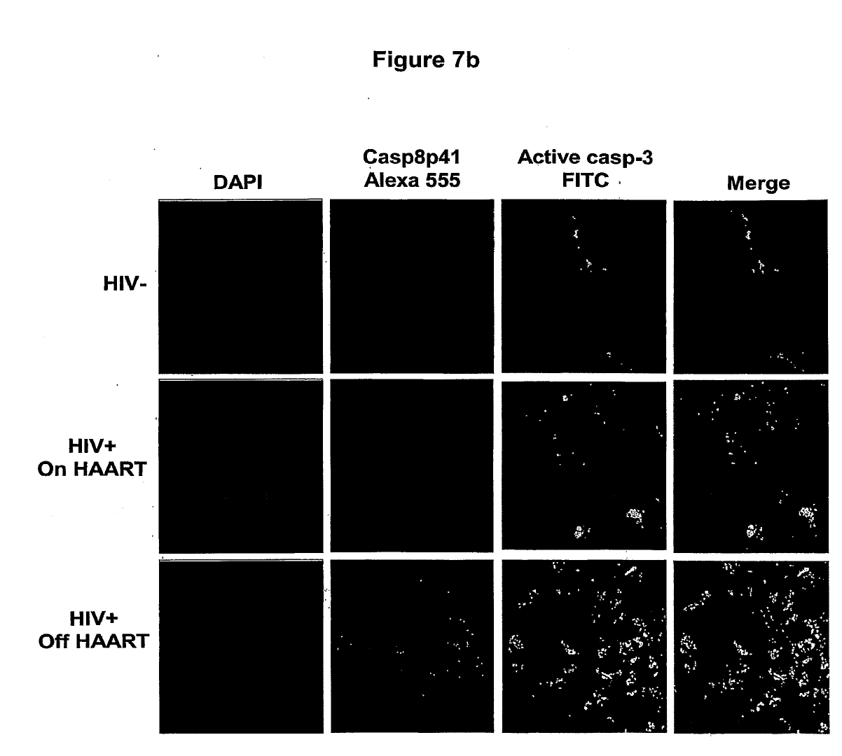

No p24 or casp8p41 reactivity was evident in tissues from RN-negative patients (FIG. 7A). In contrast, tissues from HIV-infected donors in whom viral replication was not suppressed showed diffuse and widespread p24 and casp8p41 immunoreactivity that was usually colocalized (FIG. 7A). In rare cases, however, p24-positive cells were present that were negative for casp8p41 polypeptide. In no instances were casp8p41-positive cells present that were p24 negative. In tissues of HIV-infected patients with suppressed levels of viral replication, p24 staining was rare, but colocalized with casp8p41 staining when present, indicating protease activity (FIG. 7A). Moreover, casp8p41 polypeptide was observed to colocalize with active caspase-3 polypeptide, indicating that casp8p41-positive cells are apoptotic (FIG. 7B).

### Example 8

### Linking casp8p41 Polypeptide and NFκB activation

Activation of the polypeptide signaling cascade resulting in up-regulation of NFκB dependant genes is important for HIV replication. In fact, HIV replication has been shown to be dependant on NFκB activation. NFκB activation is also an important factor in the expression of many anti-apoptotic cellular polypeptides. Therefore, an increase in activation of NFκB-mediated transcription can lead to an increase in HIV replication along with an increase in expression of polypeptides that keep the cell alive. Although HIV infection ultimately leads to cell death in the majority of cases, increases in NFκB activation can result in an increase in HIV production prior to cell death.

Using an over-expression system, caspase-8 polypeptide was found to increase NFκB activation. Importantly, casp8p41 polypeptide, the HIV protease-induced cleavage product of caspase-8 polypeptide, activated NFκB-mediated transcription two-fold more than full-length caspase-8 polypeptide. These observations indicated that HIV increases its own transcription and replication as it kills the cell it has infected.

### Example 9

### Screening HIV Positive Blood Using an anti-casp8p41 Monoclonal Antibody

The availability of an anti-casp8p41 antibody offers clinicians the opportunity to design optimal treatment therapies for their HIV patients. Initial, pretreatment flow cytometry analysis comparing the level of casp8p41 positivity with HIV p24 antigen expression in CD4⁺ cells can be helpful in determining the rate at which the virus will deplete CD4⁺ cells. A high casp8p41 polypeptide to HIV p24 polypeptide ratio may indicate a more rapid depletion of CD4⁺ cells and may warrant a more aggressive treatment approach. Conversely, a low casp8p41 polypeptide to HIV p24 polypeptide ratio may indicate a less aggressive virus, especially when coupled with a higher than usual CD4⁺ count. This may also help identify “discordant” patients who have a much better prognosis.

Within days after beginning treatment, analysis of patient blood for casp8p41 polypeptide using flow cytometry indicates the effectiveness of the therapy prescribed and, when warranted, allows for a change in medication much sooner than waiting to assess the effectiveness of the treatment based on CD4⁺ cell number. The effectiveness of an HIV treatment can also be assessed by analyzing blood samples with an ELISA using the anti-casp8p41 antibody.

The casp8p41 antibody may also be useful in designing protease inhibitors that are more effective in blocking the specific activity of HIV protease against caspase-8, which initiates apoptosis in infected cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody preparation comprising an antibody or fragment thereof, wherein said antibody or said fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide.

2. The antibody preparation of claim 1, wherein said antibody is a monoclonal antibody.

3. The antibody preparation of claim 1, wherein said antibody or said fragment thereof comprises a label selected from the group consisting of an enzyme, streptavidin, avidin, a fluorescent molecule, a luminescent molecule, a bioluminescent molecule, and a radioactive molecule.

4. An isolated cell that produces an antibody or fragment thereof, wherein said antibody or said fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide.

5. The isolated cell of claim 4, wherein said cell is a hybridoma.

6. The isolated cell of claim 4, wherein said antibody is a monoclonal antibody.

7. A method for determining whether or not a sample contains a casp8p41 polypeptide, said method comprising:
a) contacting said sample with an antibody or fragment thereof to form a complex between said casp8p41 polypeptide, if present in said sample, and said antibody or said fragment thereof, wherein said antibody or said fragment thereof binds to a polypeptide consisting of a sequence selected from the group consisting of the sequence set forth in SEQ ID NO:2 and the sequence set forth in SEQ ID NO:3 and does not bind to a human procaspase-8 polypeptide; and
b) determining the presence or absence of said complex, wherein said presence indicates that said sample contains said casp8p41 polypeptide, and wherein said absence indicates that said sample does not contain said casp8p41 polypeptide.

8. The method of claim 7, wherein said sample is blood.

9. The method of claim 7, wherein said determining step comprises performing an ELISA or flow cytometry.

10. A method for determining whether or not a treatment for an immunodeficiency virus infection decreases cell death, said method comprising determining whether or not the level of an IVP caspase polypeptide in a first sample from a mammal infected with an immunodeficiency virus is greater than or equal to the level of said IVP caspase polypeptide in a second sample from said mammal, wherein said first sample is from said mammal after said mammal received said treatment, wherein said second sample is obtained at a later time than said first sample, wherein a level of said IVP caspase polypeptide in said first sample that is equal to or greater than the level of said IVP caspase polypeptide in said second sample indicates that said treatment decreases cell death, and wherein a level of said IVP caspase polypeptide in said first sample that is lower than the level of said TVP caspase polypeptide in said second sample indicates that said treatment does not decrease cell death.

11. The method of claim 10, wherein said immunodeficiency virus is human immunodeficiency virus.

12. The method of claim 10, wherein said mammal is a human.

13. The method of claim 10, wherein said IVP caspase polypeptide is a casp8p41 polypeptide.

14. The method of claim 10, wherein said sample is blood.

15. A method for monitoring an immunodeficiency virus infection, said method comprising determining whether or not the level of an IVP caspase polypeptide in a first sample from a mammal infected with an immunodeficiency virus is greater than the level of said IVP caspase polypeptide in a second sample from said mammal, wherein said second sample is from said mammal at a time later than that of said first sample, wherein a level of said IVP caspase polypeptide in said first sample that is greater than the level of said IVP caspase polypeptide in said second sample indicates suppression of said infection, and wherein a level of said IVP caspase polypeptide in said first sample that is lower than the level of said IVP caspase polypeptide in said second sample indicates progression of said infection.

16. A method for determining the prognosis of an immunodeficiency virus infection, said method comprising determining the level of an IVP caspase polypeptide in a mammal infected with an immunodeficiency virus, wherein a detectable level of said IVP caspase polypeptide indicates a poor prognosis, and wherein and an undetectable level of said IVP caspase polypeptide indicates a good prognosis.

17. The method of claim 16, wherein said determining step comprises analyzing a sample from said mammal.

18. A method for detecting the presence of an immunodeficiency virus in a mammal, said method comprising determining whether or not an IVP caspase polypeptide is present within a mammal, wherein a detectable level of an IVP caspase polypeptide indicates the presence of an immunodeficiency virus, and wherein an undetectable level of said IVP caspase polypeptide indicates the absence of an immunodeficiency virus.

19. The method of claim 18, wherein said determining step comprises analyzing a sample from said mammal.


SEQUENCE LISTING (16 sequences)

SEQ ID NO: 1 - protein, 479 aa, Homo sapiens [sequence omitted]

SEQ ID NO: 2 - protein, 355 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEPPIYELTSQFTGLKCPSLAGKPKVF

SEQ ID NO: 3 - protein, 11 aa, Artificial Sequence
  C-terminal end of caspase-8 p41 polypeptide
CPSLAGKPKVF

SEQ ID NO: 4 - protein, 11 aa, Artificial Sequence
  Seven alanine amino acids and four c-terminal amino acids of caspase-8 p41 polypeptide
AAAAAAAPKVF

SEQ ID NO: 5 - protein, 522 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRHEDILSILTAVNDDVSRRVDKQGTKKQMPQPAFTLRKKLVFPVPLDALSL

SEQ ID NO: 6 - protein, 303 aa, Homo sapiens [sequence omitted]

SEQ ID NO: 7 - protein, 115 aa, Pan troglodytes [sequence omitted]

SEQ ID NO: 8 - protein, 263 aa, Pan troglodytes
MADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSHGEENVIYGKDGVTPIKDLTAHFRGDRCKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPRFHEKKQIPCVVSMLTKELYFSQ

SEQ ID NO: 9 - protein, 234 aa, Pan troglodytes
MKSQGQHWCSSSDKNCKVSFREKLLIIDSNLGVQDVENLKFLCIGLVPNKLLETSSSASDVFEPFLAEDLLSEDDPFFLAELLYIIRQKKLLQHLNYTKEEVERLLPTRQRVSLFRNLLYELSEGIDSENLKDMIFLLKDSLPKTEMTSLSFLAFLEKQGKIDEDNLTCLEDLCTTVVPKLLRNIEKYKREKAVQIVTPPVDKEAESYQGEEELVSQTDVKTFLEALPVGVLAK

SEQ ID NO: 10 - DNA, 48 bp, Artificial Sequence
  Oligonucleotide
cccaagcttatggactacaaagacgatgacggtaccatggacttcagc

SEQ ID NO: 11 - DNA, 68 bp, Artificial Sequence
  Oligonucleotide
ccgggcccttactacagatcctcttctgagatgagtttttgttctctagattgatcagaagggaaaag

SEQ ID NO: 12 - protein, 14 aa, Homo sapiens
RGYCLIINNHNFAK

SEQ ID NO: 13 - protein, 25 aa, Homo sapiens
KGIIYGTDGQEAPIYELTSQFTGLK

SEQ ID NO: 14 - protein, 15 aa, Homo sapiens
KVFFIQACQGDNYQK

SEQ ID NO: 15 - DNA, 2243 bp, Homo sapiens
ggacgtcgaggcattacaatcgcgaaaccaagccatagcatgaaacagcgagcttgcagcctcaccgacgagtctcaactaaaagggactcccggagctaggggtggggactcggcctcacacagtgagtgccggctattggacttttgtccagtgacagctgagacaacaaggaccacgggaggaggtgtaggagagaagcgccgcgaacagcgatcgcccagcaccaagtccgcttccaggctttcggtttctttgcctccatcttgggtgcgccttcccggcgtctaggggagcgaaggctgaggtggcagcggcaggagagtccggccgcgacaggacgaactcccccactggaaaggattctgaaagaaatgaagtcagccctcagaaatgaagttgactgcctgctggctttcctgttgactggcccggagctgtactgcaagacccttgtgagcttccctagtctaagagtaggatgtctgctgaagtcatccatcaggttgaagaagcacttgatacagatgagaaggagatgctgctctttttgtgccgggatgttgctatagatgtggttccacctaatgtcagggaccttctggatattttacgggaaagaggtaagctgtctgtcggggacttggctgaactgctctacagagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatttggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggctccataatgggagaagtaaagaacaaagacttaaggaacagcttggcgctcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctatgaagtccagaaattcttgcatctcagtatgcatggtatatcccagattcttggccaatttgcctgtatgcccgagcaccgagactacgacagctttgtgtgtgtcctggtgagccgaggaggctcccagagtgtgtatggtgtggatcagactcactcagggctccccctgcatcacatcaggaggatgttcatgggagattcatgcccttatctagcagggaagccaaagatgttttttattcagaactatgtggtgtcagagggccagctggagaacagcagcctcttggaggtggatgggccagcgatgaagaatgtggaattcaaggctcagaagcgagggctgtgcacagttcaccgagaagctgacttcttctggagcctgtgtactgcggacatgtccctgctggagcagtctcacagctcaccgtccctgtacctgcagtgcctctcccagaaactgagacaagaaagaaaacgcccactcctggatcttcacattgaactcaatggctacatgtatgattggaacagcagagtttctgccaaggagaaatattatgtctggctgcagcacactctgagaaagaaacttatcctctcctacacataagaaaccaaaaggctgggcgtagtggctcacacctgtaatcccagcactttgggaggccaaggagggcagatcacttcaggtcaggagttcgagaccagcctggccaacatggtaaacgctgtccctagtaaaaatgcaaaaattagctgggtgtgggtgtgggtacctgtgttcccagttacttgggaggctgaggtgggaggatcttttgaacccaggagttcagggtcatagcatgctgtgattgtgcctacgaatagccactgcataccaacctgggcaatatagcaagatcccatctctttaaaaaaaaaaaaaa

SEQ ID NO: 16 - protein, 480 aa, Homo sapiens
MSAEVIHQVEEALDTDEKEMLLFLCRDVAIDVVPPNVRDLLDILRERGKLSVGDLAELLYRVRRFDLLKRILKMDRKAVETHLLRNPHLVSDYRVLMAEIGEDLDKSDVSSLIFLMKDYMGRGKISKEKSFLDLVVELEKLNLVAPDQLDLLEKCLKNIHRIDLKTKIQKYKQSVQGAGTSYRNVLQAAIQKSLKDPSNNFRLHNGRSKEQRLKEQLGAQQEPVKKSIQESEAFLPQSIPEERYKMKSKPLGICLIIDCIGNETELLRDTFTSLGYEVQKFLHLSMHGISQILGQFACMPEHRDYDSFVCVLVSRGGSQSVYGVDQTHSGLPLHHIRRMFMGDSCPYLAGKPKMFFIQNYVVSEGQLENSSLLEVDGPAMKNVEFKAQKRGLCTVHREADFFWSLCTADMSLLEQSHSSPSLYLQCLSQKLRQERKRPLLDLHIELNGYMYDWNSRVSAKEKYYVWLQHTLRKKLILSYT